(19) United States
(12) Patent Application Publication
(10) Pub. No.: US 2025/0348985 A1
(43) Pub. Date: Nov. 13, 2025

(54) IMAGE PROCESSING APPARATUS, MEDICAL SYSTEM, IMAGE PROCESSING APPARATUS OPERATION METHOD, AND COMPUTER-READABLE RECORDING MEDIUM

(71) Applicant: OLYMPUS MEDICAL SYSTEMS CORP., Tokyo (JP)

(72) Inventors: Tomoya SATO, Tokorozawa-shi (JP); Takaaki IGARASHI, Tokyo (JP); Akihiro KUBOTA, Tokyo (JP); Yamato KANDA, Tokyo (JP); Yasunori MORITA, Tokyo (JP)

(73) Assignee: OLYMPUS MEDICAL SYSTEMS CORP., Tokyo (JP)

(21) Appl. No.: 19/280,587

(22) Filed: Jul. 25, 2025

Related U.S. Application Data
(63) Continuation of application No. PCT/JP2024/002309, filed on Jan. 25, 2024.

(30) Foreign Application Priority Data
Jan. 26, 2023 (WO) PCT/JP2023/002521

(51) Int. Cl. G06T 5/94; G06T 7/00

(52) U.S. Cl. CPC G06T 5/94; G06T 7/0012; G06T 2207/10068

(57) ABSTRACT

An image processing apparatus includes a processor configured to: irradiate a biological tissue including a microstructure and a microvessel with illumination light including blue-violet narrow band light, and acquire an image signal generated by capturing return light from the biological tissue; extract local contrast information in the image signal; and perform one or more of enhancement processing or suppression processing on the image signal based on the local contrast information so as to generate a display image, the enhancement processing being processing of enhancing at least one of microstructure information related to the microstructure in the biological tissue or microvessel information related to the microvessel in the biological tissue, the suppression processing being processing of suppressing at least one of the microstructure information related to the microstructure in the biological tissue or the microvessel information related to the microvessel in the biological tissue.

### CROSS-REFERENCE TO RELATED APPLICATION

This application is a continuation of International Application No. PCT/JP2024/002309, filed on Jan. 25, 2024, the entire contents of which are incorporated herein by reference.

### BACKGROUND

### 1. Technical Field

The present disclosure relates to an image processing apparatus, a medical system, an image processing apparatus operation method, and a computer-readable recording medium.

### 2. Related Art

In the related art, endoscope systems use a technique known as “Vessel Plus Surface (VS) Classification” that focuses on a fine mucosal structure in a subject in diagnosis. The “VS Classification” performs independent diagnosis on a microvascular image (microvascular pattern: V) in a mucosal surface layer and on a microsurface image (microvascular pattern: S) in a mucosa surface layer. Therefore, it is desired to perform enhanced display of both the microvascular pattern and the microsurface pattern. Accordingly, JP 6017669 B discloses a technique of applying frequency filtering to an image obtained by specialized light observation using blue-violet narrow band light beams to individually extract a glandular structure and a microvessel.

### SUMMARY

In some embodiments, an image processing apparatus includes a processor configured to: irradiate a biological tissue including a microstructure and a microvessel with illumination light including blue-violet narrow band light, and acquire an image signal generated by capturing return light from the biological tissue; extract local contrast information in the image signal; and perform one or more of enhancement processing or suppression processing on the image signal based on the local contrast information so as to generate a display image, the enhancement processing being processing of enhancing at least one of microstructure information related to the microstructure in the biological tissue or microvessel information related to the microvessel in the biological tissue, the suppression processing being processing of suppressing at least one of the microstructure information related to the microstructure in the biological tissue or the microvessel information related to the microvessel in the biological tissue.

In some embodiments, a medical system includes a light source device, an imaging device, and a medical device. The light source device includes a light source configured to irradiate a biological tissue including a microstructure and a microvessel with illumination light including blue-violet narrow band light, the imaging device includes an image sensor configured to generate an image signal by capturing return light from the biological tissue, the medical device includes a processor configured to: acquire the image signal; extract local contrast information in the image signal; perform one or more of enhancement processing or suppression processing on the image signal based on the local contrast information so as to generate a display image, the enhancement processing being processing of enhancing at least one of microstructure information related to a microstructure in the biological tissue or microvessel information related to a microvessel in the biological tissue, the suppression processing being processing of suppressing at least one of the microstructure information related to the microstructure in the biological tissue or the microvessel information related to the microvessel in the biological tissue.

In some embodiments, provided is an operation method of an image processing apparatus, the image processing apparatus including a processor, the method to be performed by the processor. The method includes: controlling a light source to emit at least blue-violet light and acquiring an image signal generated at emission of the blue-violet light; extracting local contrast information in the image signal; and performing one or more of enhancement processing or suppression processing on the image signal based on the local contrast information so as to generate a display image, the enhancement processing being processing of enhancing at least one of microstructure information related to a microstructure in a biological tissue or microvessel information related to a microvessel in the biological tissue, the suppression processing being processing of suppressing at least one of microstructure information related to the microstructure in the biological tissue or microvessel information related to the microvessel in the biological tissue.

In some embodiments, provided is a non-transitory computer-readable recording medium with an executable program stored thereon. The program causing a processor to execute: irradiating a biological tissue including a microstructure and a microvessel with illumination light including blue-violet narrow band light, and acquiring an image signal generated by capturing return light from the biological tissue; extracting local contrast information in the image signal; and performing one or more of enhancement processing or suppression processing on the image signal based on the local contrast information so as to generate a display image, the enhancement processing being processing of enhancing at least one of microstructure information related to the microstructure in the biological tissue or microvessel information related to the microvessel in the biological tissue, the suppression processing being processing of suppressing at least one of the microstructure information related to the microstructure in the biological tissue or the microvessel information related to the microvessel in the biological tissue.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic configuration diagram of an endoscope system according to a first embodiment;

FIG. 2 is a block diagram illustrating a functional configuration of a main portion of the endoscope system according to the first embodiment;

FIG. 3 is a flowchart illustrating an outline of processing executed by the endoscope system according to the first embodiment;

FIG. 4 is a diagram schematically illustrating an outline of processing executed by the endoscope system according to the first embodiment;

FIG. 5 is a block diagram illustrating a functional configuration of an endoscope system according to a second embodiment;

FIG. 6 is a flowchart illustrating an outline of processing executed by the endoscope system according to the second embodiment;

FIG. 7A is a diagram schematically illustrating an outline of enhancement processing and suppression processing executed by an adjustment unit according to the second embodiment;

FIG. 7B is a diagram schematically illustrating an outline of enhancement processing and suppression processing executed by an adjustment unit according to a first modification of the second embodiment;

FIG. 7C is a diagram schematically illustrating an outline of enhancement processing and suppression processing executed by an adjustment unit according to a second modification of the second embodiment;

FIG. 7D is a diagram of a table illustrating an example of a relationship between an input value and an output value based on FIG. 7C in each enhancement mode for each channel constituting an input image;

FIG. 7E is a diagram schematically illustrating an outline of enhancement processing and suppression processing executed by an adjustment unit according to a third modification of the second embodiment;

FIG. 7F is a diagram of a table illustrating an example of a relationship between an input value and an output value based on FIG. 7E in each enhancement degree for each channel constituting an input image;

FIG. 8 is a diagram schematically illustrating an outline of enhancement processing and suppression processing executed by an adjustment unit according to a third embodiment;

FIG. 9 is a block diagram illustrating a functional configuration of an endoscope system according to a fourth embodiment;

FIG. 10 is a flowchart illustrating an outline of processing executed by the endoscope system according to the fourth embodiment;

FIG. 11 is a diagram schematically illustrating an outline of processing executed by the endoscope system according to the fourth embodiment;

FIG. 12 is a diagram schematically illustrating an outline of enhancement processing and suppression processing executed by an adjustment unit according to the fourth embodiment;

FIG. 13 is a block diagram illustrating a functional configuration of an endoscope system according to a fifth embodiment;

FIG. 14 is a flowchart illustrating an outline of processing executed by the endoscope system according to the fifth embodiment;

FIG. 15 is a diagram schematically illustrating an outline of processing executed by the endoscope system according to the fifth embodiment;

FIG. 16 is a diagram schematically illustrating an outline of enhancement processing and suppression processing executed by an adjustment unit according to the fifth embodiment;

FIG. 17 is a diagram schematically illustrating an outline of enhancement processing and suppression processing executed by an adjustment unit according to the fifth embodiment;

FIG. 18 is a block diagram illustrating a functional configuration of an endoscope system according to a sixth embodiment;

FIG. 19 is a diagram illustrating an example of a selection setting screen on which a control unit selects the enhancement degree of a VS enhancement mode to be displayed on a display device based on an input from an input unit of the endoscope system according to the sixth embodiment;

FIG. 20 is a diagram of a parameter table illustrating an example of a relationship between the enhancement degree of a first image processing unit and the enhancement level of each enhancement type set by an enhancement processing unit of a second image processing unit to be processed for each channel constituting an input image, in the endoscope system according to the sixth embodiment;

FIG. 21 is a diagram of another parameter table illustrating an example of a relationship between the enhancement degree in each enhancement mode in the first image processing unit and each enhancement type and enhancement level set by the enhancement processing unit of the second image processing unit to be processed for each channel constituting an input image, in the endoscope system according to the sixth embodiment;

FIG. 22 is a block diagram illustrating a functional configuration of an endoscope system according to a seventh embodiment;

FIG. 23 is a diagram of a parameter table illustrating a relationship between an enhancement type set in processing mode 2 and enhancement mode/enhancement type set in processing mode 1;

FIG. 24 is a diagram illustrating an outline when switching the enhancement type in processing mode 2 and the enhancement type in processing mode 1 independently of each other;

FIG. 25 is a diagram illustrating an outline when switching the enhancement type in processing mode 2 and the enhancement type in processing mode 1 in coordination with or independently of each other;

FIG. 26 is a diagram illustrating an outline when switching the enhancement type in processing mode 2 and the enhancement type in processing mode 1 by selecting independently for each enhancement mode; and

FIG. 27 is a diagram illustrating an outline when the enhancement type in processing mode 2 and the enhancement type in processing mode 1 is switched by selecting the types independently or continuously for each enhancement mode.

### DETAILED DESCRIPTION

Hereinafter, embodiments of the present disclosure will be described in detail with reference to the drawings. The present disclosure is not limited to the following embodiments. The drawings referred to in the following description merely schematically illustrate the shapes, sizes, and positional relations to such degrees that the contents of the present disclosure are understandable. Accordingly, the present disclosure is not limited to the shapes, sizes, and positional relations exemplified in the individual drawings. In the description of the drawings, the same portions are given the same reference numerals. As an example of the endoscope system according to the present disclosure, an endoscope system including a flexible endoscope will be described.

### First Embodiment

### Configuration of Endoscope System

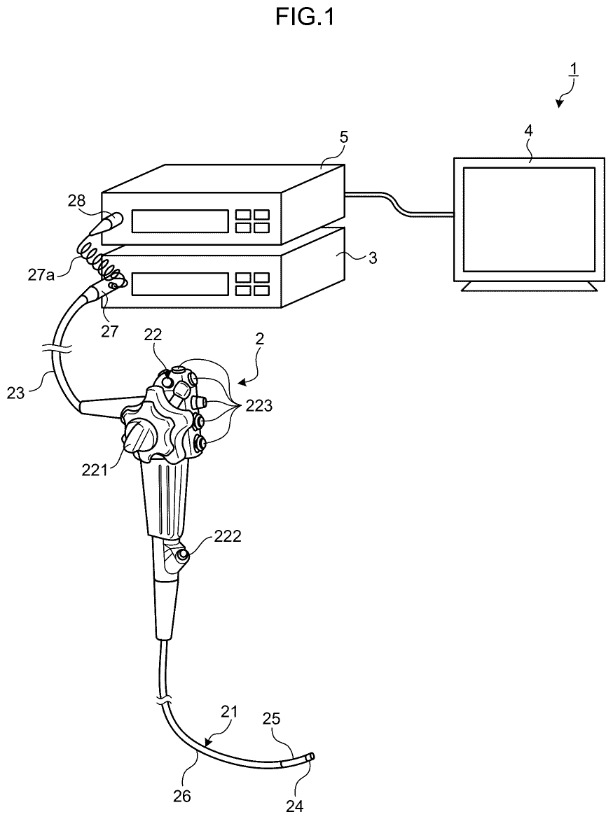

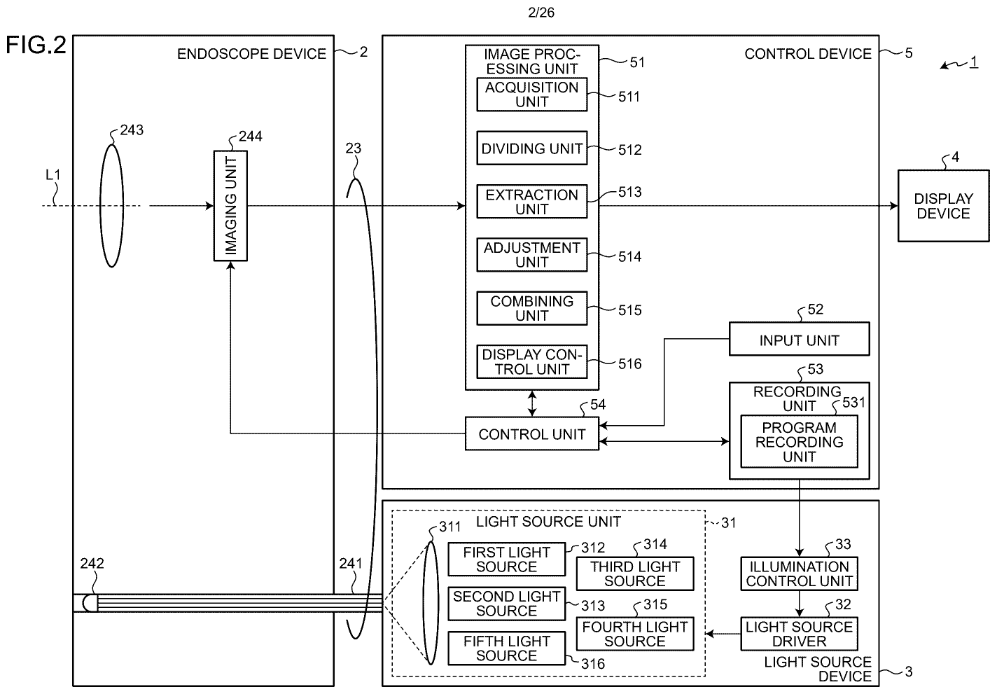

FIG. 1 is a schematic configuration diagram of an endoscope system according to a first embodiment. FIG. 2 is a block diagram illustrating a functional configuration of a main portion of the endoscope system according to the first embodiment. The endoscope system 1 illustrated in FIGS. 1 and 2 displays a display image based on an image signal (image data) generated by insertion into a body of a subject such as a patient and capturing the inside of the body of the subject. By observing the display image, a user such as a medical practitioner examines the presence or absence of a bleeding site, a tumor site, and an abnormal site, or measures the sizes of those sites. In the first embodiment, an endoscope system using the flexible endoscope illustrated in FIG. 1 will be described as the endoscope system 1. However, the system is not limited thereto, and may be, for example, a medical system including a rigid endoscope. Furthermore, the endoscope system 1 can also be implemented by adopting a medical microscope, a medical surgical robot system, or the like that performs surgery, treatment, or the like while displaying a display image based on an image signal (image data) captured by an endoscope on a display device.

An endoscope system 1 illustrated in FIG. 1 includes an endoscope device 2, a light source device 3, a display device 4, and a control device 5.

### Configuration of Endoscope Device

First, a configuration of the endoscope device 2 will be described.

The endoscope device 2 is inserted into a subject, captures an image of the inside of the subject body to generate an image signal (PAW data) and outputs the generated image signal to the control device 5. The endoscope device 2 includes an insertion unit 21, an operating unit 22, and a universal cord 23.

The insertion unit 21 has an elongated shape having flexibility. The insertion unit 21 includes: a distal end 24 incorporating an imaging unit 244 described below; a bending portion 25 being a bendable portion formed with a plurality of bending pieces; and a flexible tube 26 being a long and flexible portion connected with a proximal end of the bending portion 25.

The distal end 24 includes glass fiber or the like. The distal end 24 includes: a light guide 241 forming a light guide path of light supplied from the light source device 3; an illumination lens 242 provided at the distal end of the light guide 241; an optical system 243 that condenses at least one of reflected light and return light from the subject; and an imaging unit 244 disposed at an image forming position of the optical system 243.

The illumination lens 242 includes one or a plurality of lenses, and emits light supplied from the light guide 241 to the outside.

The optical system 243 includes one or a plurality of lenses, and condenses return light from the subject and reflected light reflected by the subject to form a subject image on an imaging surface of the imaging unit 244. The optical system 243 may have a structure capable of changing a focal position (in-focus position) by moving along an optical axis L1 under driving of an actuator (not illustrated). Of course, the optical system 243 may include a zoom lens group capable of changing the focal length by moving a plurality of lenses along the optical axis L1.

The imaging unit 244 includes an image sensor such as a Charge Coupled Device (CCD) sensor or a Complementary Metal Oxide Semiconductor (CMOS), captures an image at a predetermined frame rate to generate an image signal (RAW data), and outputs the generated image signal to the control device 5.

The operating unit 22 includes: a bending knob 221 used to bend the bending portion 25 in up-down directions and left-right directions; a treatment tool insertion unit 222 used for inserting a treatment tool such as biopsy forceps, a laser scalpel, or an inspection probe into the body cavity; and a plurality of switches 223 that receives an input of an operation instruction signal not only for the light source device 3 and the control device 5 but also for peripheral devices such as an air feeding unit, a water feeding unit, or a gas feeding unit or an input of a pre-freeze signal that instructs the imaging unit 244 to capture a still image. The treatment tool inserted through the treatment tool insertion unit 222 comes out from an aperture (not illustrated) via a treatment tool channel (not illustrated) of the distal end 24.

The universal cord 23 incorporates at least the light guide 241 and a condensing cable bundling one or a plurality of cables. The assembly cable is a signal line used for transmitting and receiving signals among the endoscope device 2, the light source device 3, and the control device 5, and includes a signal line for transmitting and receiving setting data (signal data), a signal line for transmitting and receiving an image signal (image data), a signal line for transmitting and receiving a clock signal for driving the imaging unit 244, and the like. The universal cord 23 has a connector unit 27 detachable from the light source device 3. The connector unit 27 is equipped with a coil cable 27a being an extension having a coil shape. At an extending end of the coil cable 27a, there is a connector unit 28 detachably attached to the control device 5.

### Configuration of Light Source Device

Next, a configuration of the light source device 3 will be described.

The light source device 3 supplies illumination light for irradiating the subject from the distal end 24 of the endoscope device 2. The light source device 3 includes a light source unit 31, a light source driver 32, and an illumination control unit 33.

The light source unit 31 irradiates the subject with at least one of: white light including light in a red wavelength band, light in a green wavelength band, and light in a blue wavelength band; and specialized light. The light source unit 31 includes a condenser lens 311, a first light source 312, a second light source 313, a third light source 314, a fourth light source 315, and a fifth light source 316.

The condenser lens 311 includes one or a plurality of lenses. The condenser lens 311 condenses light emitted individually from the first light source 312, the second light source 313, the third light source 314, the fourth light source 315, and the fifth light source 316, and emits the condensed light to the light guide 241.

The first light source 312 includes a red Light Emitting Diode (LED) lamp. The first light source 312 emits light in a red wavelength band (610 nm to 750 nm) (hereinafter, simply referred to as “R light”) based on the current supplied from the light source driver 32.

The second light source 313 includes a green LED lamp. The second light source 313 emits light (hereinafter, simply referred to as “G light”) in a green wavelength band (500 nm to 560 nm) based on the current supplied from the light source driver 32.

The third light source 314 includes a blue LED lamp. The third light source 314 emits light in a blue wavelength band (435 nm to 480 nm) (hereinafter, simply referred to as “B light”) based on the current supplied from the light source driver 32.

The fourth light source 315 includes a purple LED lamp. The fourth light source 315 emits narrow band light in a wavelength band of blue-violet (for example, 400 nm to 435 nm) (hereinafter, simply referred to as “V light”) based on the current supplied from the light source driver 32.

The fifth light source 316 includes: a green LED lamp; and a transmission filter that transmits a predetermined wavelength band. The fifth light source 316 emits narrow band light in a predetermined wavelength band (530 nm to 550 nm) (hereinafter, simply referred to as “NG light”) based on the current supplied from the light source driver 32.

Under the control of the illumination control unit 33, the light source driver 32 supplies a current to the first light source 312, the second light source 313, the third light source 314, the fourth light source 315, and the fifth light source 316 to cause the light sources to emit light according to the observation mode set in the endoscope system 1. Specifically, when the observation mode set in the endoscope system 1 is a normal observation mode, the light source driver 32, under the control of the illumination control unit 33, causes the first light source 312, the second light source 313, and the third light source 314 to emit white light (hereinafter, simply referred to as “W light”). When the observation mode set in the endoscope system 1 is a specialized light observation mode, the light source driver 32, under the control of the illumination control unit 33, causes the fourth light source 315 and the fifth light source 316 to emit specialized light (hereinafter, simply referred to as “S light”) capable of performing Narrow Band Imaging (NBI).

The illumination control unit 33 controls the lighting timing of the light source device 3 based on an instruction signal received from the control device 5. Specifically, the illumination control unit 33 causes the first light source 312, the second light source 313, and the third light source 314 to emit light at a predetermined period. The illumination control unit 33 includes a central processing unit (CPU), or the like. Furthermore, in a case where the observation mode of the endoscope system 1 is the normal observation mode, the illumination control unit 33 controls the light source driver 32 to cause the first light source 312, the second light source 313, and the third light source 314 to emit W light. Furthermore, in a case where the observation mode of the endoscope system 1 is the specialized light observation mode, the illumination control unit 33 controls the light source driver 32 to combine the fourth light source 315 and the fifth light source 316 to emit S light. The illumination control unit 33 may control the light source driver 32 in accordance with the observation mode of the endoscope system 1 to cause any two or more of the first light source 312, the second light source 313, the third light source 314, the fourth light source 315, and the fifth light source 316 to emit light in combination.

### Configuration of Display Device

Next, a configuration of the display device 4 will be described.

The display device 4 displays a display image based on the image data generated by the endoscope device 2 and received from the control device 5. Moreover, the display device 4 displays various types of information related to the endoscope system 1. The display device 4 includes a display panel of liquid crystal, organic electroluminescence (EL), or the like.

### Configuration of Control Device

Next, a configuration of the control device 5 will be described.

The control device 5 receives the image data generated by the endoscope device 2, performs predetermined image processing on the received image data, and outputs the processed image data to the display device 4. In addition, the control device 5 integrally controls the entire operation of the endoscope system 1. The control device 5 includes an image processing unit 51, an input unit 52, a recording unit 53, and a control unit 54.

Under the control of the control unit 54, the image processing unit 51 acquires the image signal generated by the endoscope device 2, performs predetermined image processing on the acquired image signal, and outputs the processed image signal to the display device 4. The image processing unit 51 includes memory and a processor having hardware such as a Graphics Processing Unit (GPU), a Digital Signal Processing (DSP) chip, or a Field Programmable Gate Array (FPGA). The image processing unit 51 includes an acquisition unit 511, a dividing unit 512, an extraction unit 513, an adjustment unit 514, a combining unit 515, and a display control unit 516.

The acquisition unit 511 acquires an image signal (PAW data) from the imaging unit 244 of the endoscope device 2. Specifically, the imaging unit 244 irradiates biological tissue including a microstructure and a microvessel with illumination light including blue-violet narrow band light and captures return light from the biological tissue, and then, the acquisition unit 511 acquires an image signal generated by the capturing.

The dividing unit 512 divides the input image corresponding to the image signal acquired by the acquisition unit 511 into an illumination light component and a reflectance component. Specifically, the dividing unit 512 divides the input image corresponding to the image signal into a base image which is an illumination light component being a low-frequency component and a detail image which is a reflectance component.

The extraction unit 513 extracts local contrast information in the image signal acquired by the acquisition unit 511. Specifically, the extraction unit 513 extracts the detail image obtained by the division performed by the dividing unit 512 as local contrast information of the reflectance component.

Based on the local contrast information extracted by the extraction unit 513, the adjustment unit 514 performs, on the image signal acquired by the acquisition unit 511, any one or more of: enhancement processing of enhancing at least one of microstructure information related to a microstructure and microvessel information related to a microvessel, in a biological tissue; and suppression processing of suppressing at least one of microstructure information related to a microstructure and microvessel information related to a microvessel, in a biological tissue.

The combining unit 515 combines the illumination light component, which is the base image divided by the dividing unit 512 and has undergone tone compression, and the reflectance component, which is a detail image that has undergone enhancement processing performed by the adjustment unit 514.

The display control unit 516 generates a display image based on a combining result obtained by the combining performed by the combining unit 515, and outputs the generated display image to the display device 4.

The input unit 52 receives an input of an instruction signal instructing the operation of the endoscope system 1 and an instruction signal instructing the observation mode of the endoscope system 1, and outputs the received instruction signals to the control unit 54. The input unit 52 includes a switch, a button, and a touch panel.

The recording unit 53 records various programs executed by the endoscope system 1, data being currently executed by the endoscope system 1, and image data generated by the endoscope device 2. The recording unit 53 includes volatile memory, nonvolatile memory, and a memory card. The recording unit 53 includes a program recording unit 531 that records various programs executed by the endoscope system 1.

The control unit 54 includes memory and a processor including at least one or more pieces of hardware such as an FPGA or a CPU. The control unit 54 controls each unit constituting the endoscope system 1.

### Processing of Endoscope System

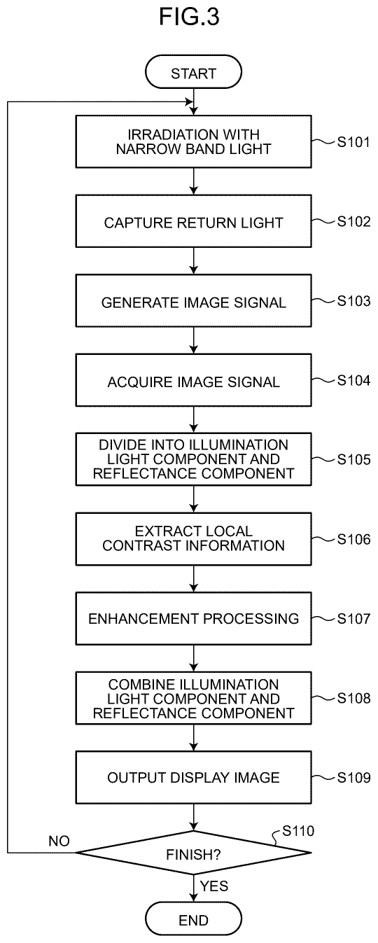

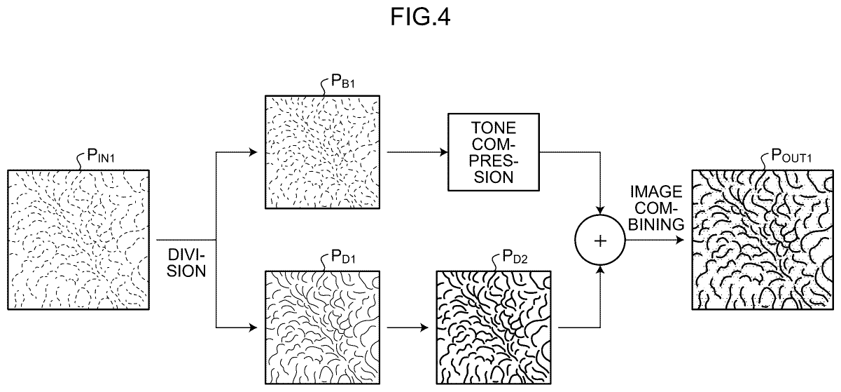

Next, processing executed by the endoscope system 1 will be described. FIG. 3 is a flowchart illustrating outline of processing executed by the endoscope system 1. FIG. 4 is a diagram schematically illustrating an outline of processing executed by the endoscope system.

As illustrated in FIG. 3, the control unit 54 first controls the illumination control unit 33 to cause the fourth light source 315 and the fifth light source 316 of the light source device 3 to emit light and irradiate the biological tissue with blue-violet and green beams of narrow band light (Step S101).

Subsequently, the control unit 54 causes the imaging unit 244 to capture the return light from the biological tissue (Step S102) and causes the imaging unit 244 to generate an image signal (Step S103).

Thereafter, the acquisition unit 511 acquires an image signal (RAW data) from the imaging unit 244 of the endoscope device 2 (Step S104).

Subsequently, the dividing unit 512 divides an input image corresponding to the image signal acquired by the acquisition unit 511 into an illumination light component and a reflectance component (Step S105). Specifically, as illustrated in FIG. 4, the dividing unit 512 divides an input image P_{IN1} corresponding to the image signal into a base image P_{B1} which is an illumination light component being a low-frequency component and a detail image P_{D1} which is a reflectance component. In this case, the dividing unit 512 applies a known bilateral filter to the input image P_{IN1}, for example, to divide the base image P_{B1}, which is the illumination light component being the low-frequency component, from the input image P_{IN1}. In this case, the dividing unit 512 performs tone compression on the base image P_{B1} and outputs the processed base image P_{B1}. In addition, based on a Retinex model, the dividing unit 512 divides the input image P_{IN1} to obtain the detail image P_{D1}, which is a reflectance component, from the input image P_{IN1}. For example, the dividing unit 512 performs division to obtain the detail image P_{D1}, which is a reflectance component, from the input image P_{IN1} based on a known Single-Scale Retinex (SSR) model in the Retinex model. Here, SSR is a technique of smoothing a target pixel and a surrounding pixel of the target pixel with a Gaussian filter to estimate the illumination light component and obtaining a reflectance component from a ratio between an input pixel value of the target pixel and the estimated illumination light component. Since the bilateral filter and the SSR are well-known techniques, detailed description thereof will be omitted.

Subsequently, the extraction unit 513 extracts the detail image obtained by the division performed by the dividing unit 512 as local contrast information of the reflectance component (Step S106). Specifically, the extraction unit 513 extracts a difference between base image P_{B1} and input image P_{IN1}, namely, the detail image, as local contrast information. In this case, based on a signal value of the target pixel in each of base image P_{B1} and input image P_{IN1} and on the signal value of each of a plurality of surrounding pixels in the target pixel, the extraction unit 513 extracts a contrast value, which is a relative signal strength ratio, as the contrast information for each pixel, thereby extracting local contrast information.

Thereafter, based on the local contrast information extracted by the extraction unit 513, the adjustment unit 514 performs enhancement processing of enhancing individual information of the microstructure information related to the microstructure and the microvessel information related to the microvessel, in the biological tissue, on the image signal acquired by the acquisition unit 511 (Step S107). In this case, based on the local contrast information extracted by the extraction unit 513, the adjustment unit 514 performs enhancement processing of enhancing a detail component on the image signal acquired by the acquisition unit 511. Specifically, as illustrated in FIG. 4, based on the local contrast information extracted by the extraction unit 513, the adjustment unit 514 performs enhancement processing of enhancing a detail component on the detail image obtained by the division performed by the dividing unit 512 to generate a detail image P_{D2}. That is, based on the local contrast information extracted by the extraction unit 513, the adjustment unit 514 performs enhancement processing of increasing a signal amplitude value of the reflectance component obtained by the division performed by the dividing unit 512.

Thereafter, the combining unit 515 combines the illumination light component, which is the base image divided by the dividing unit 512 and has undergone tone compression, and the reflectance component, which is a detail image that has undergone enhancement processing performed by the adjustment unit 514 (Step S108). Specifically, as illustrated in FIG. 4, the combining unit 515 combines the base image P_{B1} and the detail image P_{D2}.

Subsequently, the display control unit 516 generates a display image based on a combining result obtained by the combining performed by the combining unit 515, and outputs the generated display image to the display device 4 (Step S109). Specifically, as illustrated in FIG. 4, the display control unit 516 generates a display image P_{OUT1} based on the combining result generated by the combining unit 515, and outputs the generated display image P_{OUT1} to the display device 4. With this configuration, the user can improve the diagnosis accuracy by selectively enhancing the feature data of the display image.

Subsequently, the control unit 54 determines whether an instruction signal of instructing an end of observation of the subject has been input from the input unit 52 (Step S110). When the control unit 54 determines that the instruction signal of instructing the end of the observation of the subject has been input from the input unit 52 (Step S110: Yes), the endoscope system 1 ends the present processing. In contrast, when the control unit 54 determines that the instruction signal instructing the end of the observation of the subject has not been input from the input unit 52 (Step S110: No), the endoscope system 1 returns to Step S101 described above.

According to the first embodiment described above, the combining unit 515 combines the illumination component which is the base image obtained by the division performed by the dividing unit 512 and the reflectance component which is the detail image that has undergone the enhancement processing performed by the adjustment unit 514, and the display control unit 516 generates a display image based on the combined result obtained by combining performed by the combining unit 515 and outputs the generated display image to the display device 4. This makes it possible to appropriately perform enhancement and suppression of individual portions of the microstructure and the microvessel in the image regardless of the observation distance between the distal end 24 of the endoscope device 2 and the biological tissue. This configuration makes it possible to enhance individual portions of the mucosa and the blood vessel of the biological tissue in the display image, enabling the user to facilitate focusing on a desired region (region of interest).

### Second Embodiment

Next, a second embodiment will be described. The endoscope system according to the second embodiment has a difference from the endoscope system 1 according to the above-described first embodiment in configuration and processing to be executed. Specifically, in the second embodiment, at least one of enhancement processing or suppression processing is performed based on local contrast information for each pixel. Accordingly, the following will describe a functional configuration of the endoscope system according to the second embodiment and then describe processing to be executed by the endoscope system according to the second embodiment. A same reference sign will be given to the configuration identical to the configuration of the endoscope system 1 according to the above-described first embodiment, and detailed description thereof will be omitted.

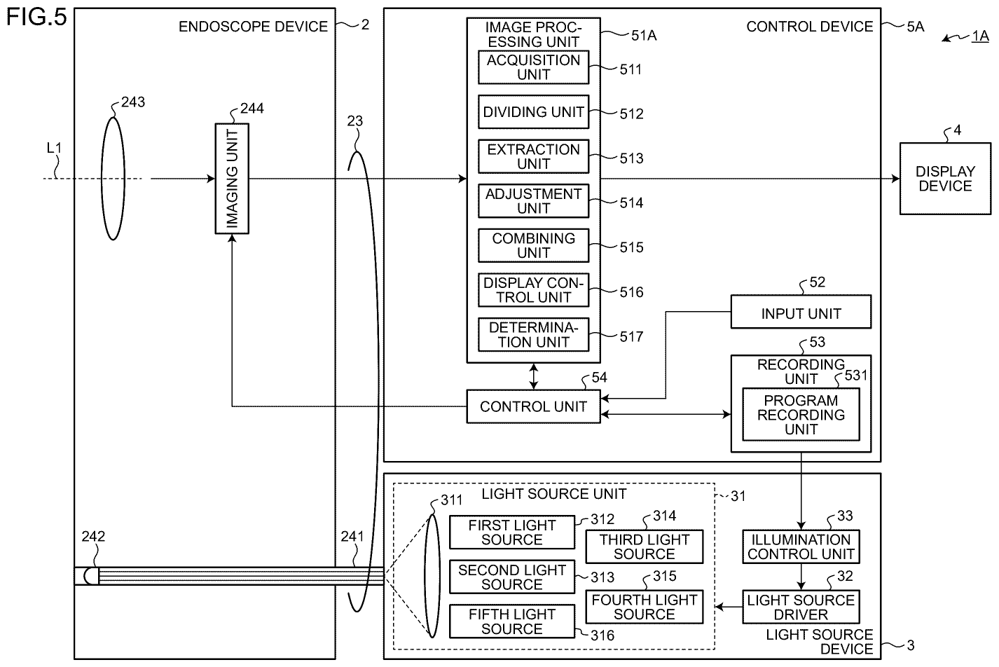

Functional configuration of endoscope system FIG. 5 is a block diagram illustrating a functional configuration of an endoscope system according to a second embodiment. An endoscope system 1A illustrated in FIG. 5 includes a control device 5A instead of the control device 5 of the endoscope system 1 according to the first embodiment described above. The control device 5A includes an image processing unit 51A instead of the image processing unit 51 according to the first embodiment described above.

The image processing unit 51A further includes a determination unit 517 in addition to the configuration of the image processing unit 51 according to the above-described first embodiment.

Based on the local contrast information extracted by the extraction unit 513, the determination unit 517 determines, for each pixel, whether the local contrast value is equal to or larger than a predetermined reference value preset, and extracts the microstructure information and the microvessel information.

### Processing of Endoscope System

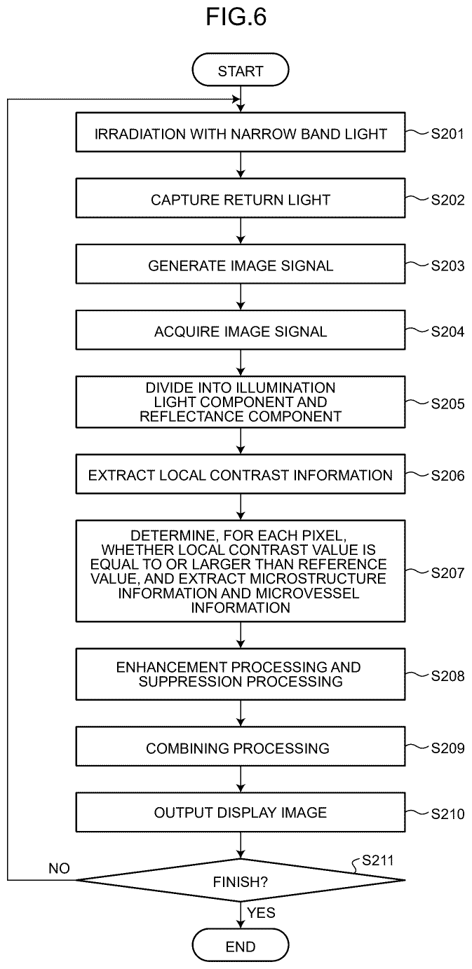

Next, processing executed by the endoscope system 1A will be described. FIG. 6 is a flowchart illustrating outline of processing executed by the endoscope system 1A. In FIG. 6, Steps S201 to S206 are similar to Steps S101 to S106 in FIG. 3 described above, and thus detailed description thereof is omitted.

In Step S207, based on the local contrast information extracted by the extraction unit 513, the determination unit 517 determines, for each pixel, whether the local contrast value is equal to or larger than a predetermined reference value preset, and extracts microstructure information and microvessel information. In the second embodiment, the determination unit 517 performs the determination using one reference value. However, the determination is not limited thereto, and it is also allowable to provide two reference values for individually extracting the microstructure information and the microvessel information.

Subsequently, based on the image signal acquired by the acquisition unit 511, the local contrast information extracted by the extraction unit 513, and the determination result obtained by the determination unit 517, the adjustment unit 514 performs at least one of: enhancement processing of enhancing at least one of microstructure information related to a microstructure and microvessel information related to a microvessel, in a biological tissue; and suppression processing of suppressing at least one of microstructure information related to a microstructure and microvessel information related to a microvessel, in a biological tissue (Step S208). Specifically, based on the image signal acquired by the acquisition unit 511 and the local contrast information extracted by the extraction unit 513, the adjustment unit 514 performs enhancement processing of setting the local contrast value away from the reference value on the pixel having a signal value determined by the determination unit 517 to have the local contrast value equal to or larger than the reference value, and performs suppression processing of bringing the local contrast value closer to the reference value on the pixel having a signal value determined by the determination unit 517 to have the local contrast value not equal to or larger than the reference value (pixel having the local contrast value smaller than the reference value).

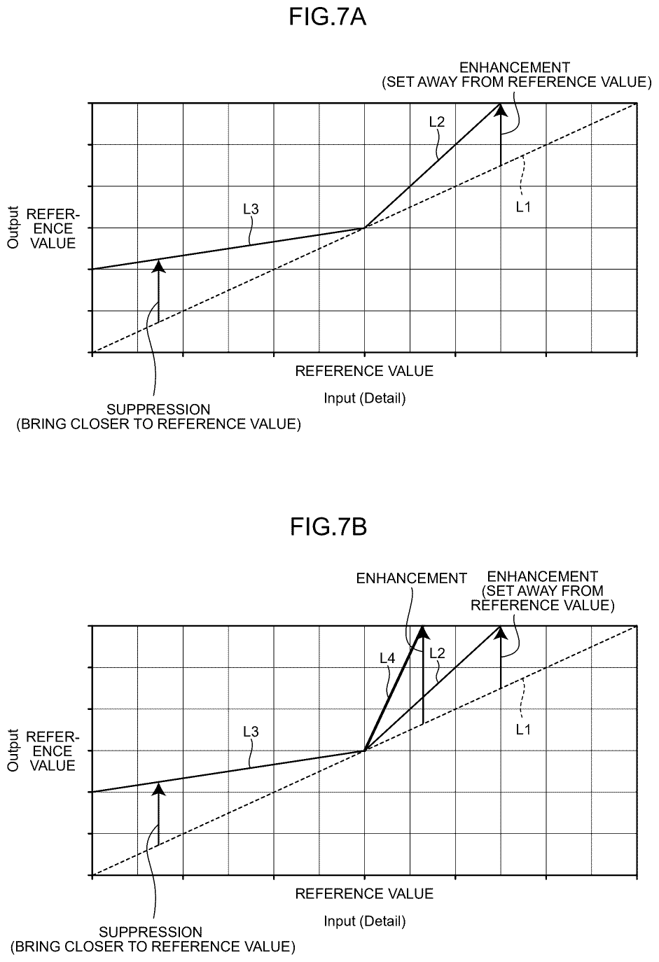

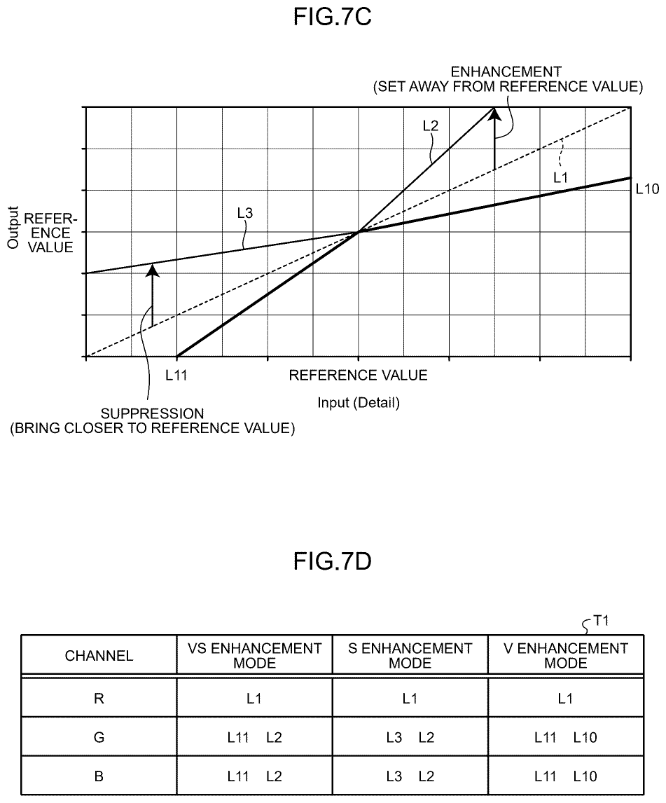

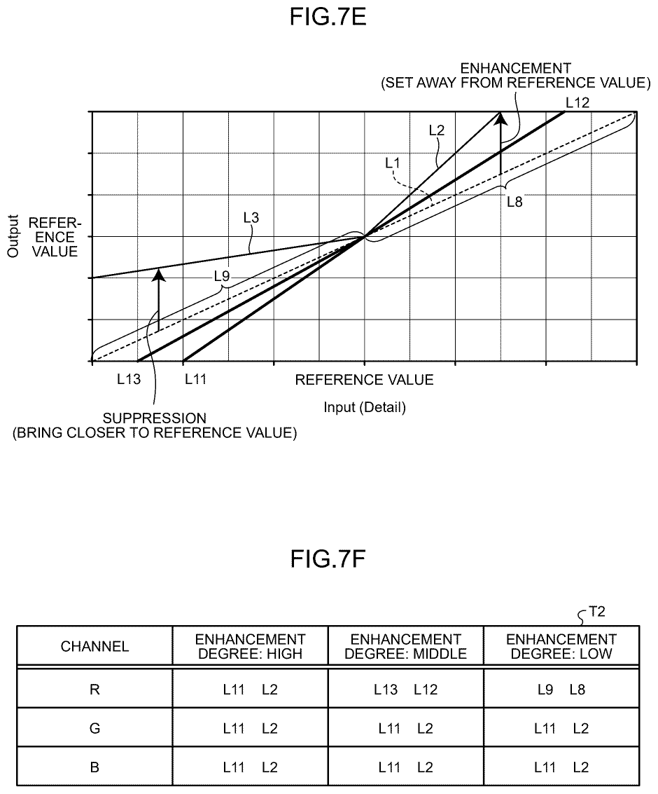

FIG. 7A is a diagram schematically illustrating an outline of enhancement processing and suppression processing executed by the adjustment unit 514. In FIG. 7A, a straight line L1 indicates a relationship between an input value and an output value of the local contrast value before adjustment, a straight line L2 indicates a relationship between the input value and the output value of the local contrast value after adjustment in a case where the local contrast value is equal to or larger than the reference value, and a straight line L3 indicates a relationship between the input value and the output value of the local contrast value after adjustment in a case where the local contrast value is not equal to or larger than the reference value.

As indicated by the straight lines L1 and L2 in FIG. 7A, based on the image signal acquired by the acquisition unit 511 and the local contrast information extracted by the extraction unit 513, the adjustment unit 514 performs enhancement processing of setting the local contrast value away from the reference value on the signal value (luminance value) of the pixel determined by the determination unit 517 to have the local contrast value equal to or larger than the reference value. That is, based on the local contrast information extracted by the extraction unit 513, the adjustment unit 514 performs enhancement processing of increasing a signal amplitude value of the reflectance component obtained by the division performed by the dividing unit 512.

In contrast, as indicated by the straight lines L1 and L3 in FIG. 7A, based on the image signal acquired by the acquisition unit 511 and the local contrast information extracted by the extraction unit 513, the adjustment unit 514 performs suppression processing of bringing the local contrast value closer to the reference value on the signal value (luminance value) of the pixel determined by the determination unit 517 to have the local contrast value not equal to or larger than the reference value. That is, the adjustment unit 514 performs suppression processing of decreasing the signal amplitude value of the illumination light component based on the local contrast information extracted by the extraction unit 513.

In this manner, the adjustment unit 514 can enhance the microstructure in the biological tissue and can suppress the microvessel in the biological tissue.

Since Steps S209 to S211 are similar to Steps S108 to S110 in FIG. 3 described above, detailed description thereof is omitted.

According to the second embodiment described above, based on the image signal acquired by the acquisition unit 511 and the local contrast information extracted by the extraction unit 513, the adjustment unit 514 performs enhancement processing of setting the local contrast value away from the reference value on the signal value of the pixel determined by the determination unit 517 to have the local contrast value equal to or larger than the reference value. Furthermore, based on the image signal acquired by the acquisition unit 511 and the local contrast information extracted by the extraction unit 513, the adjustment unit 514 performs suppression processing of bringing the local contrast value closer to the reference value on the signal value of the pixel determined by the determination unit 517 to have the local contrast value not equal to or larger than the reference value (pixel having the local contrast value smaller than the reference value). This makes it possible to selectively perform enhancement and suppression of individual portions of the mucosa as microstructure and the microvessel in the image regardless of the observation distance between the distal end 24 of the endoscope device 2 and the biological tissue.

Moreover, according to the second embodiment, based on the image signal acquired by the acquisition unit 511 and the local contrast information extracted by the extraction unit 513, the adjustment unit 514 performs enhancement processing of setting the local contrast value away from the reference value on the signal value of the pixel determined by the determination unit 517 to have the local contrast value equal to or larger than the reference value. Furthermore, based on the image signal acquired by the acquisition unit 511 and the local contrast information extracted by the extraction unit 513, the adjustment unit 514 performs suppression processing of bringing the local contrast value closer to the reference value on the signal value of the pixel determined by the determination unit 517 to have the local contrast value not equal to or larger than the reference value (pixel having the local contrast value smaller than the reference value). This prevents overexposure of the mucosa being a microstructure, making it possible for the user to easily observe the microstructure of the mucosa.

According to the second embodiment, the adjustment unit 514 performs the suppression processing of decreasing the signal amplitude value of the illumination light component based on the local contrast information extracted by the extraction unit 513. However, the processing is not limited thereto, and it is also allowable to perform, for example, enhancement processing of increasing the signal amplitude value of the illumination light component based on the local contrast information extracted by the extraction unit 513.

### First Modification of Second Embodiment

Next, a first modification the second embodiment will be described. The first modification of the second embodiment has a difference only in the processing of enhancement processing and suppression processing executed by the adjustment unit 514. Accordingly, enhancement processing and suppression processing performed by the adjustment unit 514 according to the first modification of the second embodiment will be described below. A same reference sign will be given to the configuration identical to the configuration of the endoscope system 1A according to the above-described second embodiment, and detailed description thereof will be omitted.

FIG. 7B is a diagram schematically illustrating an outline of enhancement processing and suppression processing executed by the adjustment unit 514 according to the first modification of the second embodiment. In FIG. 7B, a straight line L1 indicates a relationship between an input value and an output value of the local contrast value before adjustment, a straight line L2 indicates a relationship between the input value and the output value of the local contrast value after adjustment in a case where the local contrast value is equal to or larger than the reference value, a straight line L3 indicates a relationship between the input value and the output value of the local contrast value after adjustment in a case where the local contrast value is not equal to or larger than the reference value, and a straight line L4 indicates a relationship between the input value and the output value of the local contrast value after adjustment in a case where the local contrast value is equal to or larger than the reference value.

As indicated by the straight lines L1 and L4 in FIG. 7B, based on the image signal acquired by the acquisition unit 511 and the local contrast information extracted by the extraction unit 513, the adjustment unit 514 performs enhancement processing of setting the local contrast value away from the reference value on the signal value (luminance value) of the pixel determined by the determination unit 517 to have the local contrast value equal to or larger than the reference value. That is, based on the local contrast information extracted by the extraction unit 513, the adjustment unit 514 performs enhancement processing of increasing a signal amplitude value of the reflectance component obtained by the division performed by the dividing unit 512. Furthermore, under the control of the control unit 54, the adjustment unit 514 performs enhancement processing corresponding to individual lines of the straight lines L2 and L4 based on a selection signal input by the user such as a medical practitioner by operating the input unit 52 to select an enhancement processing mode according to the characteristic of the target disease for each patient.

According to the first modification of the second embodiment described above, the parameters of the adjustment unit 514 can be changed in accordance with the characteristic of the target disease, making it possible to generate an enhanced image according to the target disease.

### Second Modification of Second Embodiment

Next, second modification the second embodiment will be described. The second modification of the second embodiment has a difference only in the processing of enhancement processing and suppression processing executed by the adjustment unit 514. Specifically, the second modification of the second embodiment has a difference in processing of the enhancement processing and the suppression processing executed by the adjustment unit 514 and a difference in image information to be reproduced for each RGB channel input to the image processing unit 51A. Accordingly, the adjustment unit 514 executes the enhancement processing and the suppression processing using parameter switching in which the processing parameters of the enhancement modes (V enhancement mode, S enhancement mode, and VS enhancement mode) for the microstructure information and the microvessel information are switched for each RGB channel. Accordingly, enhancement processing and suppression processing performed by the adjustment unit 514 according to the second modification of the second embodiment will be described below. A same reference sign will be given to the configuration identical to the configuration of the endoscope system 1A according to the above-described second embodiment, and detailed description thereof will be omitted.

FIG. 7C is a diagram schematically illustrating an outline of enhancement processing and suppression processing executed by the adjustment unit 514 according to the first modification of the second embodiment. FIG. 7D is a diagram of a table illustrating an example of a relationship between an input value and an output value based on FIG. 7C in each enhancement mode for each channel constituting an input image. A straight line L1 indicates a relationship between an input value and an output value of the local contrast value before adjustment, a straight line L2 indicates a relationship between the input value and the output value of the local contrast value after adjustment in a case where the local contrast value is equal to or larger than the reference value, a straight line L3 indicates a relationship between the input value and the output value of the local contrast value after adjustment in a case where the local contrast value is not equal to or larger than the reference value, a straight line L10 indicates a relationship between the input value and the output value of the local contrast value after adjustment in a case where the local contrast value is equal to or larger than the reference value, and a straight line L11 indicates a relationship between the input value and the output value of the local contrast value after adjustment in a case where the local contrast value is not equal to or larger than the reference value. In addition, table T1 illustrated in FIG. 7D is preliminarily recorded in the recording unit 53.

As illustrated in the straight lines L1 to L3, L10, and L11 in FIG. 7C and table T1 in FIG. 7D, under the control of the control unit 54, the adjustment unit 514 performs enhancement processing and suppression processing for each RGB channel in accordance with processing parameters of outputting an output value based on an input value of a local contrast value for each enhancement mode (V enhancement mode, S enhancement mode, or VS enhancement mode) based on a selection signal input by the user such as a medical practitioner by operating the input unit 52 to select an enhancement mode according to the characteristic of the target disease for each patient.

According to the second modification of the second embodiment described above, the parameters of the adjustment unit 514 can be changed in accordance with the characteristics of the target disease, making it possible to generate an image that has undergone enhancement processing and suppression processing in which the relationship between the input value and the output value of the local contrast value has been changed for each RGB channel in accordance with the target disease.

### Third Modification of Second Embodiment

Next, a third modification the second embodiment will be described. The third modification of the second embodiment has a difference only in the processing of enhancement processing and suppression processing executed by the adjustment unit 514. Specifically, the third modification of the second embodiment has a difference in processing of the enhancement processing and the suppression processing executed by the adjustment unit 514 and a difference in image information to be reproduced for each RGB channel input to the image processing unit 51A. Accordingly, the adjustment unit 514 executes the enhancement processing and the suppression processing using parameter switching in which the processing parameters of the enhancement modes (V enhancement mode, S enhancement mode, and VS enhancement mode) for the microstructure information and the microvessel information are switched for each RGB channel. Accordingly, enhancement processing and suppression processing performed by the adjustment unit 514 according to the second modification of the second embodiment will be described below. A same reference sign will be given to the configuration identical to the configuration of the endoscope system 1A according to the above-described second embodiment, and detailed description thereof will be omitted.

FIG. 7E is a diagram schematically illustrating an outline of enhancement processing and suppression processing executed by the adjustment unit 514 according to the third modification of the second embodiment. FIG. 7F is a diagram of a table illustrating an example of a relationship between an input value and an output value based on FIG. 7E in each enhancement degree for each channel constituting an input image.

A straight line L1 indicates a relationship between an input value and an output value of the local contrast value before adjustment, a straight line L2 indicates a relationship between the input value and the output value of the local contrast value after adjustment in a case where the local contrast value is equal to or larger than the reference value, a straight line L3 indicates a relationship between the input value and the output value of the local contrast value after adjustment in a case where the local contrast value is not equal to or larger than the reference value, a straight line L8 is a part of the straight line L1 and indicates a relationship between the input value and the output value of the local contrast value after adjustment in a case where the local contrast value is equal to or larger than the reference value, a straight line L9 is a part of the straight line L1 and indicates a relationship between the input value and the output value of the local contrast value after adjustment in a case where the local contrast value is not equal to or larger than the reference value, and a straight line L11 indicates a relationship between the input value and the output value of the local contrast value after adjustment in a case where the local contrast value is not equal to or larger than the reference value, and a straight line L12 indicates a relationship between the input value and the output value of the local contrast value after adjustment in a case where the local contrast value is equal to or larger than the reference value. Furthermore, a straight line L13 indicates a relationship between the input value and the output value of the local contrast value after adjustment in a case where the local contrast value is not equal to or larger than the reference value. In addition, table T2 illustrated in FIG. 7F is prerecorded in the recording unit 53.

As illustrated in the straight lines L1 to L3 and L11 to L13 or L8 and L9 in FIG. 7E and table T2 in FIG. 7F, under the control of the control unit 54, the adjustment unit 514 executes the enhancement processing and the suppression processing such that, when the enhancement degrees in an identical enhancement mode are different in a case of executing a selected enhancement mode based on the selection signal input by the user such as a medical practitioner by operating the input unit 52 to select the enhancement mode selected in accordance with the characteristic of the target disease for each patient, the adjustment unit 514 executes the enhancement processing and the suppression processing with the settings in which the relationship between the input value and the output value of the local contrast value is set to be different among the R channel, the G channel, and the B channel.

According to the third modification of the second embodiment described above, the parameters of the adjustment unit 514 can be changed in accordance with the characteristics of the target disease, making it possible to generate an image that has undergone enhancement processing and suppression processing in which the relationship between the input value and the output value of the local contrast value has been changed for each RGB channel for the target disease even in a case where the enhancement degrees within an identical enhancement mode are different.

### Third Embodiment

Next, a third embodiment will be described. An endoscope system according to the third embodiment has the same configuration as the endoscope system 1A according to the second embodiment described above, in which individual processing of the enhancement processing and the suppression processing performed by the adjustment unit 514 has a difference. Accordingly, enhancement processing and suppression processing performed by the adjustment unit 514 will be described below. A same reference sign will be given to the configuration identical to the configuration of the endoscope system 1A according to the above-described second embodiment, and detailed description thereof will be omitted.

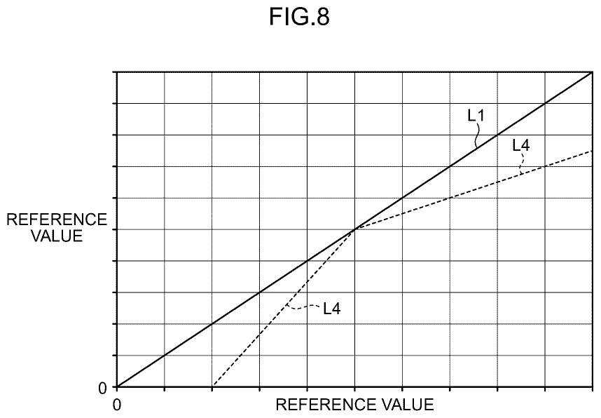

FIG. 8 is a diagram schematically illustrating an outline of enhancement processing and suppression processing executed by the adjustment unit 514 according to the third embodiment. In FIG. 8, a straight line L1 indicates a relationship between the input value and the output value of the local contrast value before adjustment, and a straight line L4 indicates a relationship between the input value and the output value of the local contrast value after adjustment in a case where the local contrast value is not equal to or larger than the reference value.

As illustrated by the straight lines L1 and L4 in FIG. 8, based on the detail image P_{D1} obtained by the division performed by the dividing unit 512 and the local contrast information of the detail image P_{D1} extracted by the extraction unit 513, the adjustment unit 514 performs at least one of enhancement processing of enhancing and suppression processing of suppressing individual information of the microstructure information related to the microstructure in the biological tissue and the microvessel information related to the microvessel.

Specifically, as illustrated in FIG. 8, based on the detail image P_{D1} obtained by the division performed by the dividing unit 512 and the local contrast information of the detail image P_{D1} extracted by the extraction unit 513, the adjustment unit 514 performs enhancement processing of setting the local contrast value away from the reference value on the signal value of the pixel determined by the determination unit 517 to have the local contrast value equal to or larger than the reference value, and performs suppression processing of bringing the local contrast value closer to the reference value on the signal value of the pixel determined by the determination unit 517 to have the local contrast value not equal to or larger than the reference value (pixel having the local contrast value smaller than the reference value).

For example, the adjustment unit 514 performs suppression processing of setting the local contrast value away from the reference value nonlinearly with a gentle inclination on the signal value of the pixel determined by the determination unit 517 to have the local contrast value not equal to or larger than the reference value (pixel having the local contrast value smaller than the reference value), and performs suppression processing of achieving linear output at a prescribed signal value. With this configuration, the blood vessel is enhanced as compared with the mucosa, making it possible for the user to improve the diagnosis accuracy based on the blood vessel structure.

According to the third embodiment described above, based on the image signal acquired by the acquisition unit 511 and the local contrast information extracted by the extraction unit 513, the adjustment unit 514 performs enhancement processing of setting the local contrast value away from the reference value on the signal value of the pixel determined by the determination unit 517 to have the local contrast value equal to or larger than the reference value. Furthermore, based on the image signal acquired by the acquisition unit 511 and the local contrast information extracted by the extraction unit 513, the adjustment unit 514 performs suppression processing of bringing the local contrast value closer to the reference value on the signal value of the pixel determined by the determination unit 517 to have the local contrast value not equal to or larger than the reference value (pixel having the local contrast value smaller than the reference value). This makes it possible to selectively perform enhancement and suppression of individual portions of the mucosa as microstructure and the microvessel in the image regardless of the observation distance between the distal end 24 of the endoscope device 2 and the biological tissue.

### Fourth Embodiment

Next, a fourth embodiment will be described. The endoscope system according to the fourth embodiment has a difference from the endoscope system 1A according to the above-described second embodiment in configuration and processing to be executed. Specifically, the endoscope system 1A according to the second embodiment described above combines an unprocessed base image with the detail image that has undergone at least one of the enhancement processing or the suppression processing, whereas the endoscope system according to the fourth embodiment performs predetermined image processing on the base image and then combines the processed base image with the detail image. Accordingly, the following will describe a configuration of the endoscope system according to the fourth embodiment and then describe processing to be executed by the endoscope system. A same reference sign will be given to the configuration identical to the configuration of the endoscope system 1A according to the above-described second embodiment, and detailed description thereof will be omitted.

### Functional Configuration of Endoscope System

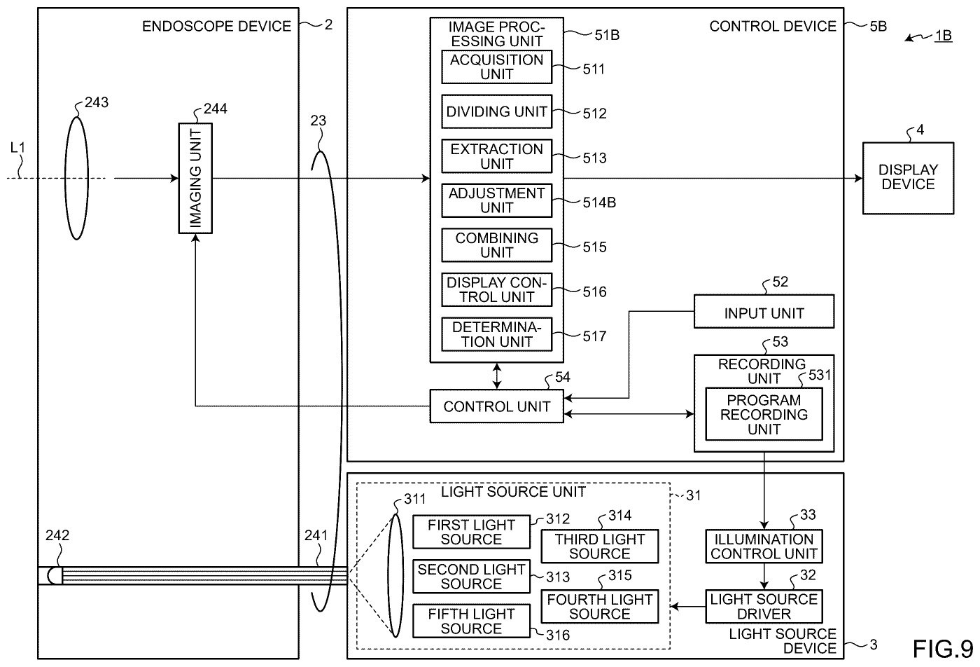

FIG. 9 is a block diagram illustrating a functional configuration of an endoscope system according to the fourth embodiment. An endoscope system 1B illustrated in FIG. 9 includes a control device 5B instead of the control device 5A of the endoscope system 1A according to the second embodiment described above. The control device 5B includes an image processing unit 51B instead of the image processing unit 51A according to the second embodiment described above. The image processing unit 51B includes an adjustment unit 514B instead of the adjustment unit 514 according to the second embodiment described above.

Based on the image signal acquired by the acquisition unit 511 and the local contrast information extracted by the extraction unit 513, the adjustment unit 514B performs at least one of enhancement processing of enhancing and suppression processing of suppressing individual information of microstructure information related to a microstructure in the biological tissue and microvessel information related to a microvessel. Furthermore, the adjustment unit 514B performs gain adjustment processing of performing gain adjustment on the base image which is a illumination light component divided by the dividing unit 512.

### Processing of Endoscope System

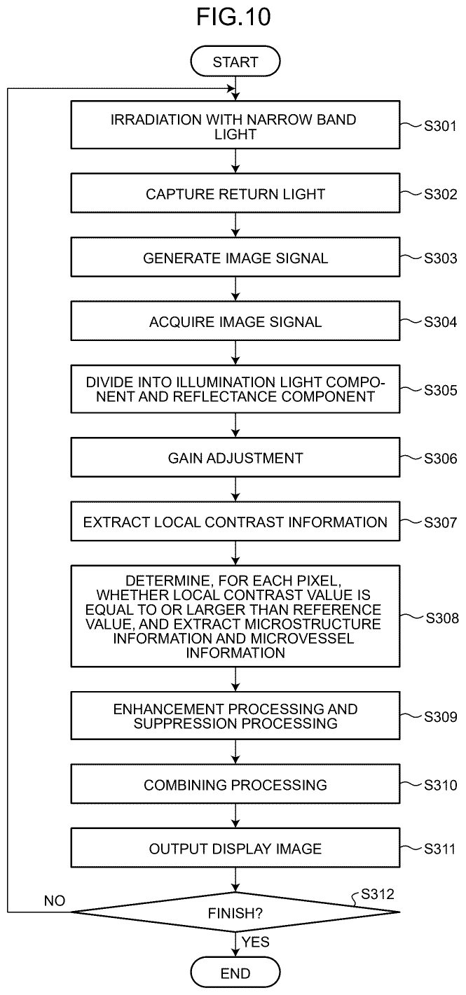

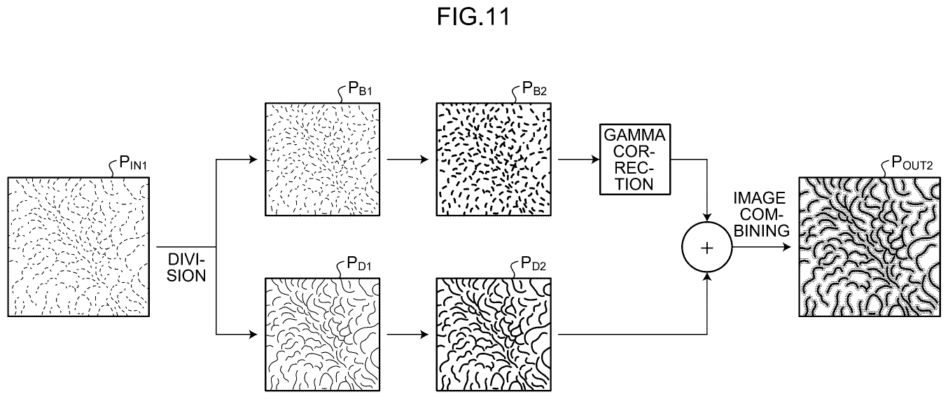

FIG. 10 is a flowchart illustrating outline of processing executed by the endoscope system 1B. FIG. 11 is a diagram schematically illustrating an outline of processing executed by the endoscope system 1B. In FIG. 10, Steps S301 to S305 are similar to Steps S101 to S105 in FIG. 3 described above, and thus detailed description thereof is omitted.

In Step S306, the adjustment unit 514B performs gain adjustment processing of performing gain adjustment on the base image P_{B1}, which is the illumination light component obtained by the division performed by the dividing unit 512. Specifically, as illustrated in FIG. 11, the adjustment unit 514B performs gain adjustment processing of reducing the gain on the base image P_{B1} to generate a base image P_{B2}. For example, the adjustment unit 514B multiplies the signal value of each pixel constituting the base image P_{B1} by 0.8 to generate the base image P_{B2}. That is, the adjustment unit 514B performs suppression processing of decreasing the signal amplitude value of the illumination light component.

Since Steps S307 and S308 are similar to Steps S206 and S207 in FIG. 6 described above, detailed description thereof is omitted.

In Step S309, as illustrated in FIG. 11, the adjustment unit 514B performs processing on the detail image P_{D1} to generate the detail image P_{D2}, specifically by performing enhancement processing of setting the local contrast value away from the reference value on the signal value of the pixel determined by the determination unit 517 to have the local contrast value equal to or larger than the reference value and by performing suppression processing of bringing the local contrast value closer to the reference value on the signal value of the pixel determined by the determination unit 517 to have the local contrast value not equal to or larger than the reference value. After Step S309, the endoscope system 1B proceeds to Step S310.

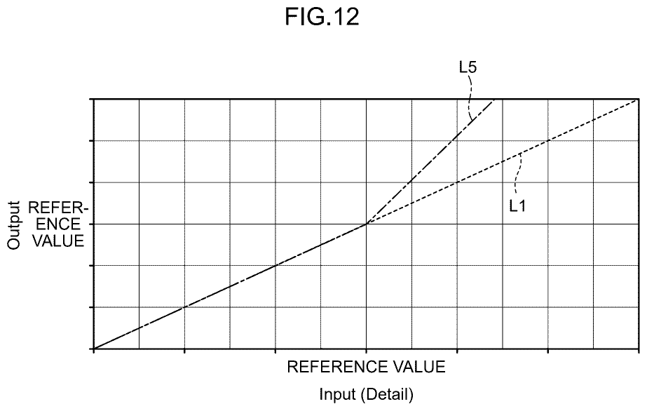

FIG. 12 is a diagram schematically illustrating an outline of enhancement processing and suppression processing executed by the adjustment unit 514B. In FIG. 10, a straight line L1 indicates a relationship between the input value and the output value of the local contrast value before adjustment, and a polygonal line L5 indicates a relationship between the input value and the output value of the local contrast value after adjustment.

As indicated by the straight line L1 and the polygonal line L5 in FIG. 12, the adjustment unit 514B performs, on the detail image P_{D1}, enhancement processing, specifically processing of suppressing halation by performing an enhancement of higher intensity than the enhancement in the second embodiment described above on a signal value of the pixel having a local contrast value higher than the reference value by a predetermined value or more while setting the local contrast value away from the reference value on the signal value of a pixel determined by the determination unit 517 to have the local contrast value equal to or larger than the reference value. Furthermore, the adjustment unit 514B performs suppression processing of setting the local contrast value away from the reference value nonlinearly with a gentle inclination on the signal value of the pixel determined by the determination unit 517 to have the local contrast value not equal to or larger than the reference value (pixel having the local contrast value smaller than the reference value), being the signal value near the reference value, thereby generating the detail image P_{D2}. In this case, as illustrated by a polygonal line L5 in FIG. 12, the adjustment unit 514B performs enhancement processing and suppression processing, being processing of enhancing the signal value such that the slope of the coefficient to be multiplied by the signal value is to be equal to or larger than the reference value. With this processing, the adjustment unit 514 can enhance the microstructure in the biological tissue and can suppress the microvessel in the biological tissue.

Subsequently, the combining unit 515 combines the base image that has undergone the gain adjustment processing performed by the adjustment unit 514B and the detail image that has undergone at least one of the enhancement processing or the suppression processing performed by the adjustment unit 514 (Step S310). Specifically, as illustrated in FIG. 11, the combining unit 515 combines the base image P_{B2} and the detail image P_{D2}.

Thereafter the display control unit 516 generates a display image based on a combining result obtained by the combining performed by the combining unit 515, and outputs the generated display image to the display device 4 (Step S311). Specifically, as illustrated in FIG. 11, the display control unit 516 generates a display image P_{OUT2} based on the combining result generated by the combining unit 515, and outputs the generated display image P_{OUT2} to the display device 4.

Since Step S312 is similar to Step S110 in FIG. 3 described above, its description will be omitted.

According to the fourth embodiment described above, the adjustment unit 514B performs gain adjustment processing of performing gain adjustment on the base image P_{B1}, which is the illumination light component obtained by the division performed by the dividing unit 512. In addition, the adjustment unit 514B performs enhancement processing and suppression processing on the detail image, being the reflectance component obtained by the division performed by the dividing unit 512, to generate the detail image, specifically by performing enhancement processing of setting the local contrast value away from the reference value on the signal value of the pixel determined by the determination unit 517 to have the local contrast value equal to or larger than the reference value, and by performing suppression processing of bringing the local contrast value closer to the reference value on the signal value of the pixel determined by the determination unit 517 to have the local contrast value not equal to or larger than the reference value. Thereafter, the combining unit 515 combines the base image that has undergone the gain adjustment processing performed by the adjustment unit 514B and the detail image that has undergone at least one of the enhancement processing or the suppression processing performed by the adjustment unit 514, making it possible to selectively perform enhancement and suppression individually on the microstructures and the microvessels in the image regardless of the observation distance.

### Fifth Embodiment

Next, a fifth embodiment will be described. The endoscope system according to the fifth embodiment has a difference from the endoscope system 1A according to the above-described second embodiment in configuration and processing to be executed. Specifically, the endoscope system according to the fifth embodiment generates two detail images (detail components) individually for a mucosa and a blood vessel so as to be combined with each other, and thereafter performs at least one of enhancement processing or suppression processing on the mucosa and the blood vessel individually. Accordingly, the following will describe a configuration of the endoscope system according to the fifth embodiment and then describe processing to be executed by the endoscope system. A same reference sign will be given to the configuration identical to the configuration of the endoscope system 1A according to the above-described second embodiment, and detailed description thereof will be omitted.

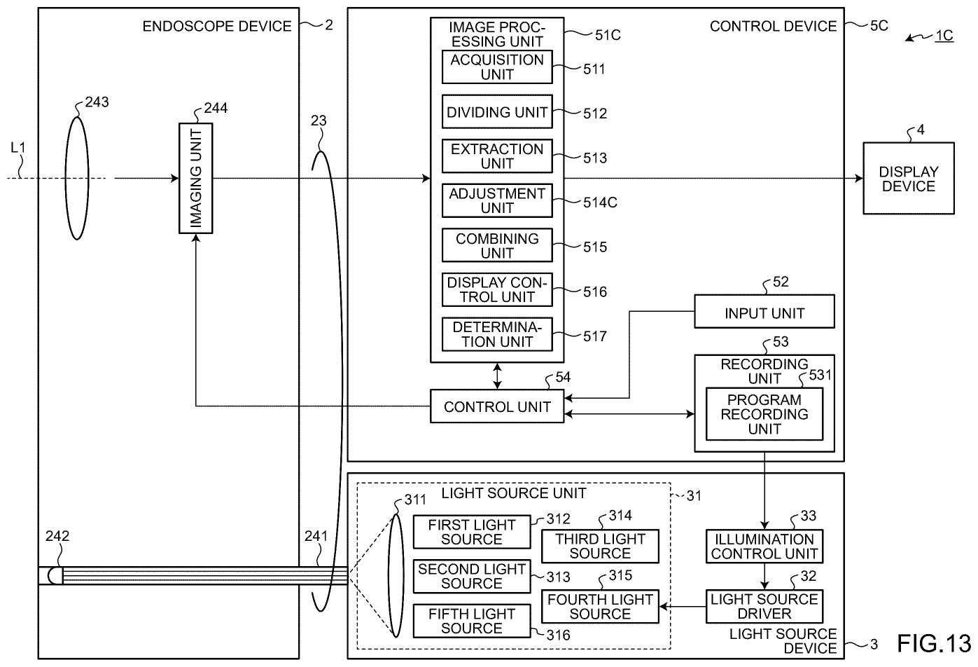

Functional configuration of endoscope system FIG. 13 is a block diagram illustrating a functional configuration of an endoscope system according to the fifth embodiment. An endoscope system 1C illustrated in FIG. 13 includes a control device 5C instead of the control device 5A of the endoscope system 1A according to the second embodiment described above. The control device 5C includes an image processing unit 51C instead of the image processing unit 51C according to the first embodiment described above. The image processing unit 51C includes an adjustment unit 514C instead of the adjustment unit 514 according to the second embodiment described above.

The adjustment unit 514C performs processing, on the bright light component obtained by the division performed by the dividing unit 512, to generate a first base component returned to the contrast of the image signal and a second base component with a reduced contrast. Furthermore, by combining the reflectance component obtained by the division performed by the dividing unit 512 with the first base component and the second base component individually, the adjustment unit 514C generates a first detail component and a second detail component different from each other. Furthermore, the adjustment unit 514C combines the first detail component and the second detail component with a predetermined coefficient to generate a third detail component, and then performs at least one of enhancement processing and suppression processing on the third detail component to generate a fourth detail component.

### Processing of Endoscope System

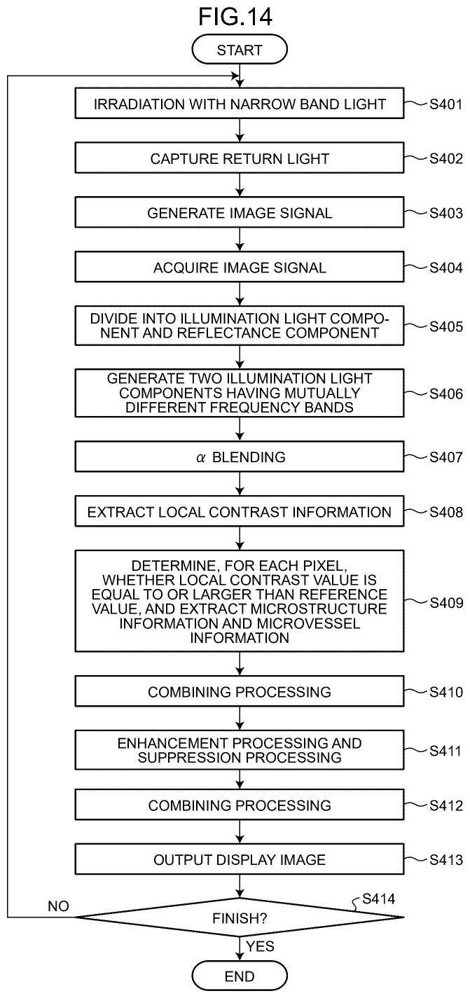

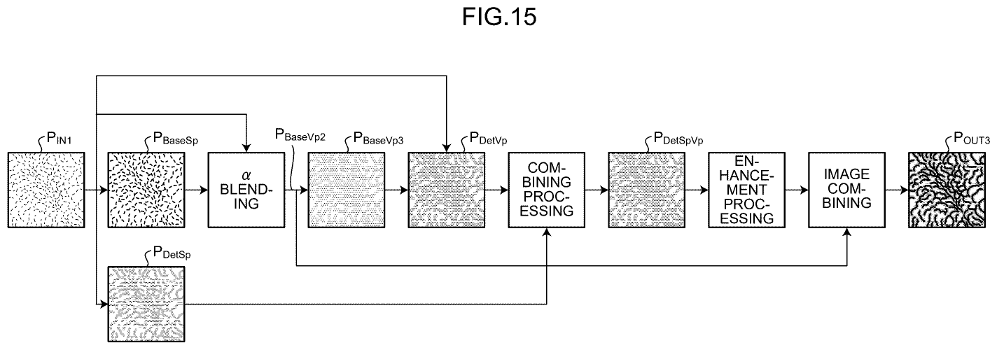

Next, processing executed by the endoscope system 1C will be described. FIG. 14 is a flowchart illustrating outline of processing executed by the endoscope system 1C. FIG. 15 is a diagram schematically illustrating an outline of processing executed by the endoscope system 1C. In FIG. 14, Steps S401 to S405 are similar to Steps S101 to S105 in FIG. 3 described above, and thus detailed description thereof is omitted.

In Step S406, based on the input image P_{IN1}, the adjustment unit 514C generates two illumination light components having mutually different frequency bands. Specifically, as illustrated in FIG. 15, based on the input image P_{IN1}, the adjustment unit 514C generates a base image P_{BaseSp} and a first detail image P_{DetSp} which are two illumination light components having mutually different frequency bands. In this case, in a case where the component of the input image P_{IN1} is I, the Gaussian is G, and the variance is σVP<σSp, the adjustment unit 514C generates the base image P_{Base}Sp by the following formulas (1) and (2).

$$Base image {P}_{BaseSp} = {G}_{VP} * I \qquad ( 1 )$$

Here, when the weight of the bilateral filter is Wbi, the base image P_{BaseSp} can be expressed by the following Formula (2).

$$Base image {P}_{BaseSp} = Wbi * I \qquad ( 2 )$$

Subsequently, the adjustment unit 514C executes alpha blending (α blending) of combining an input image P_{IN1} and a base image P_{Base} with a predetermined coefficient to generate a second detail image (Step S407).

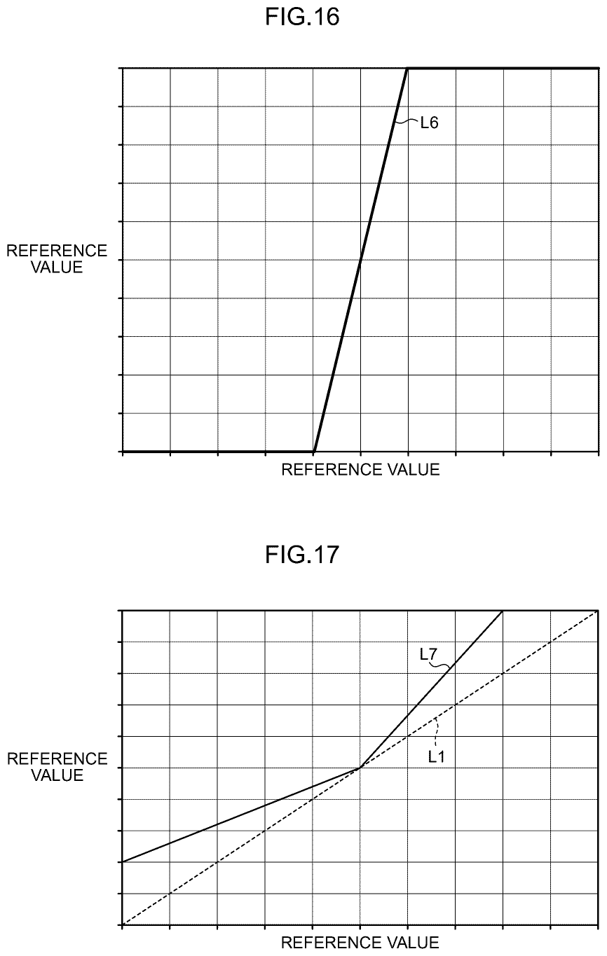

Specifically, as illustrated by a polygonal line L6 in FIGS. 15 and 16, the adjustment unit 514C performs alpha blending (a blending) using each image of the base image P_{BaseSp} and the input image P_{IN1} to generate a base image P_{BaseVp2} being an illumination light component. Specifically, the adjustment unit 514 generates the base image P_{Basevp2} by the following Formula (3).

$${P}_{BaseVp 2} = α * {P}_{IN 1} + ( 1 - α ) * {P}_{BaseSp} \qquad ( 3 )$$

α can be mixed at a constant ratio.

Since Steps S408 and S409 are similar to Steps S206 and S207 in FIG. 6 described above, detailed description thereof is omitted.

In Step S410, the adjustment unit 514C executes combining processing of combining a detail image P_{DetVp} and the detail image P_{DetSp} to generate a combined image, in which the detail image P_{DetVp} is a reflectance component obtained by combining a base image _{BaseVp3} and the input image P_{IN1}, and the detail image P_{DetSp} is an image generated in Step S406. Specifically, as illustrated in FIG. 14, the adjustment unit 514C combines the detail image P_{DetSp} with the detail image P_{DetSp} generated in Step S406, thereby generating a combined image P_{DetSpVp}.

Subsequently, the adjustment unit 514C performs, on the combined image P_{DetSpVp}, at least one of enhancement processing of enhancing or suppression processing of suppressing each information of microstructure information being information related to a microstructure, and microvessel information being information related to a microvessel, in the biological tissue (Step S411).

FIG. 17 is a diagram schematically illustrating an outline of enhancement processing and suppression processing executed by the adjustment unit 514C. In FIG. 17, a straight line L1 indicates a relationship between the input value and the output value of the local contrast value before adjustment, and a polygonal line L7 indicates a relationship between the input value and the output value of the local contrast value after adjustment.

As indicated by the polygonal line L7 in FIG. 17, based on the local contrast information extracted by the extraction unit 513, the adjustment unit 514C performs enhancement processing of setting the local contrast value away from the reference value on the signal value of the pixel determined by the determination unit 517 to have the local contrast value of the detail image P_{Detspvp} equal to or larger than the reference value, and performs suppression processing of bringing the local contrast value closer to the reference value on the signal value of the pixel determined by the determination unit 517 to have the local contrast value of the detail image P_{DetSpVp} not equal to or larger than the reference value (pixel having the local contrast value smaller than the reference value), thereby generating the detail image P_{DetSpVp}.

In Step S412, the combining unit 515 executes combining processing, being processing of combining the detail image P_{DetSpVp} generated by the adjustment unit 514C and the base image P_{BaseVp2}. Specifically, as illustrated in FIG. 15, the combining unit 515 combines the detail image P_{DetSpVp} and the base image P_{Basevp2}. Although the combining unit 515 combines the detail image P_{DetSpVp} generated by the adjustment unit 514C and the base image P_{Basevp2}, it is also allowable to combine the input image P_{IN1} instead of the base image P_{Basevp2}.

Thereafter, the display control unit 516 generates a display image based on a combining result obtained by the combining performed by the combining unit 515, and outputs the generated display image to the display device 4 (Step S413). Specifically, as illustrated in FIG. 15, the display control unit 516 generates a display image P_{OUT3} based on the combining result generated by the combining unit 515, and outputs the generated display image P_{OUT3} to the display device 4.

Since Step S414 is similar to Step S110 in FIG. 3 described above, its description will be omitted.

According to the fifth embodiment described above, it is possible to appropriately enhance each portion of the microstructure and the microvessel in the image regardless of the observation distance.

### Sixth Embodiment

Next, a sixth embodiment will be described. The endoscope system according to the sixth embodiment has a difference from the endoscope system 1A according to the above-described second embodiment in configuration and processing to be executed. Accordingly, the following will describe a functional configuration of the endoscope system according to the sixth embodiment and then describe processing to be executed by the endoscope system according to the sixth embodiment. A same reference sign will be given to the configuration identical to the configuration of the endoscope system 1A according to the above-described second embodiment, and detailed description thereof will be omitted.

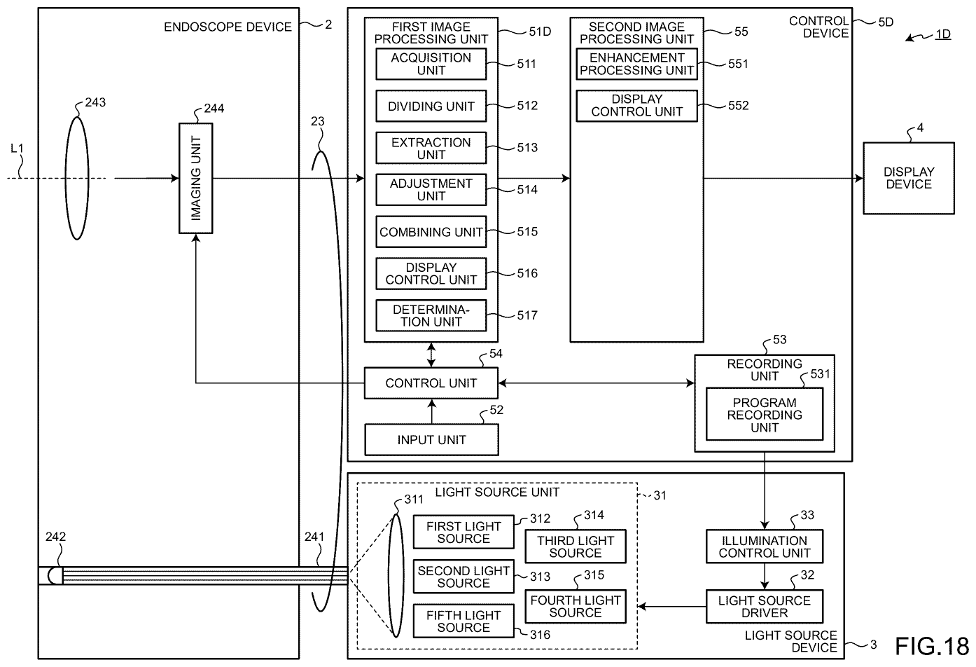

Functional configuration of endoscope system FIG. 18 is a block diagram illustrating a functional configuration of an endoscope system according to the sixth embodiment. An endoscope system 1D illustrated in FIG. 18 includes a control device 5D instead of the control device 5A of the endoscope system 1A according to the second embodiment described above. The control device 5D includes a first image processing unit 51D instead of the image processing unit 51A according to the second embodiment described above. Furthermore, the control device 5D further includes a second image processing unit 55.

The first image processing unit 51D has the same function as the image processing unit 51A according to the second embodiment described above, and includes an acquisition unit 511, a dividing unit 512, an extraction unit 513, an adjustment unit 514, a combining unit 515, a display control unit 516, and a determination unit 517.

The second image processing unit 55 includes: an enhancement processing unit 551 that performs, on the image signal that has undergone image processing performed by the first image processing unit 51D, a plurality of types of sharpness enhancement processing (for example, spatial filtering or the like) that enhances sharpness with different enhancement characteristics, and color enhancement processing (for example, IHb color enhancement that highlights a slight change in color in a mucosa) that performs color enhancement; and a display control unit 552 that generates a display image based on the image signal that has undergone image processing performed by the enhancement processing unit 551 and outputs the generated display image to the display device 4.

In the endoscope system 1D with this configuration, based on the input from the input unit 52, the control unit 54 reads processing parameters of the first image processing unit 51D and the second image processing unit 55 from the recorded data recorded by the recording unit 53 and changes the processing parameters in accordance with the enhancement degree in the enhancement mode (V enhancement mode, S enhancement mode, and VS enhancement mode) of the first image processing unit 51D and the enhancement level in the enhancement type (sharpness enhancement type A, Type B, and color enhancement type C) of the second image processing unit 55. The processing parameters of the enhancement mode are similar to those of the third modification of the second embodiment described above, and thus detailed description thereof will be omitted (refer to FIGS. 7E and 7F).

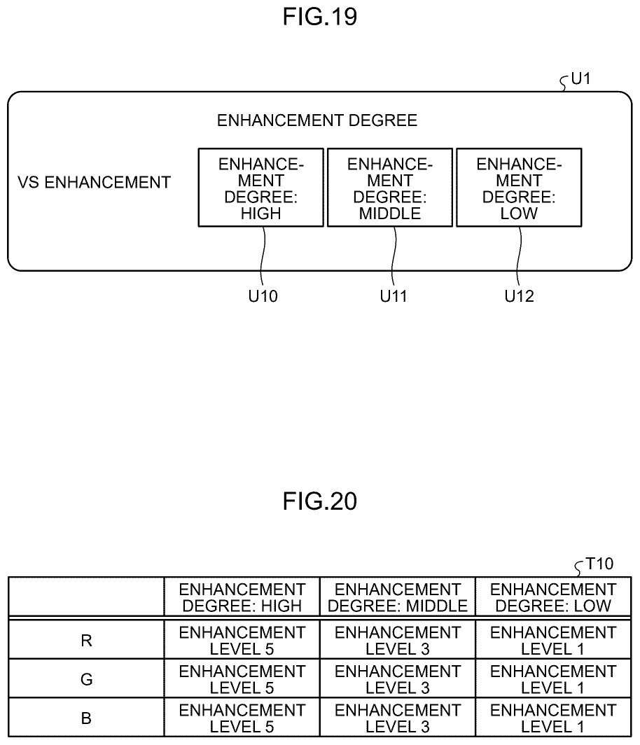

FIG. 19 is a diagram illustrating an example of a selection setting screen on which the control unit 54 selects the enhancement degree of the VS enhancement mode to be displayed on the display device 4 based on an input from an input unit 52. FIG. 20 is a diagram of a parameter table illustrating an example of a relationship between the enhancement degree of the first image processing unit 51D and the enhancement level of each enhancement type set by the enhancement processing unit 551 of the second image processing unit 55 to be processed for each channel constituting an input image.

As illustrated in FIG. 19, the user such as a medical practitioner operates the input unit 52, and selects a desired enhancement degree from a high icon U10, a middle icon U11, and a low icon U12 that instruct the enhancement degree in the VS enhancement mode on a selection setting screen U1 displayed on the display device by the control unit 54 and the display control unit 551 based on the input from the input unit 52. In response to this operation, the control unit 54 reads the processing parameters of the first image processing unit 51D and the second image processing unit 55 (refer to parameter table T10 in FIG. 20) from the recorded data recorded by the recording unit 53 and changes the processing parameters in accordance with the enhancement degree in each enhancement mode (V enhancement mode, S enhancement mode, and VS enhancement mode) of the first image processing unit 51D and the enhancement level in each enhancement type of the second image processing unit 55. That is, the control unit 54 switches the enhancement level of the second image processing unit 55 in coordination with the enhancement degree of the first image processing unit 51D.

According to the sixth embodiment described above, by switching a plurality of enhancement degrees having different degrees of enhancement, it is possible to perform observation and diagnosis with an enhancement effect appropriate for the user.

Although the sixth embodiment includes FIG. 19 illustrating the VS enhancement mode as an example, it is similarly allowable, in the V enhancement mode and the S enhancement mode, to display a selection setting screen for selecting the degree of enhancement for the V enhancement mode and the S enhancement mode, and to read and change the processing parameter with reference to parameter table T10 based on the selected enhancement degree to switch between a plurality of enhancement degrees having different degrees of enhancement.

In the sixth embodiment, the enhancement level of the enhancement type by the second image processing unit 55 is used in coordination with the enhancement degree in each enhancement mode (V enhancement mode, S enhancement mode, and VS enhancement mode) of the first image processing unit 51D. However, at least one of the enhancement type and the enhancement level may be used for the switching in coordination.

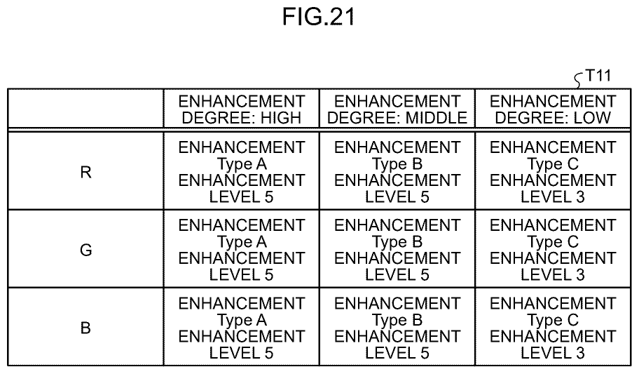

FIG. 21 is a diagram of another parameter table illustrating an example of a relationship between the enhancement degree in each enhancement mode in the first image processing unit 51D and each enhancement type and enhancement level set by the enhancement processing unit 551 of the second image processing unit 55 to be processed for each channel constituting an input image.

As illustrated in parameter table T1l in FIG. 21, the control unit 54 reads the processing parameters of the first image processing unit 51D and the second image processing unit 55 (refer to parameter table T1l in FIG. 21) from the recorded data recorded by the recording unit 53 and changes the processing parameters in accordance with the enhancement degree in each enhancement mode of the first image processing unit 51D and the enhancement level in each enhancement type by the second image processing unit 55 (sharpness enhancement Type A, Type B, and color enhancement processing Type C). That is, the control unit 54 switches the enhancement type and enhancement level of the second image processing unit 55 in coordination with the enhancement degree of the first image processing unit 51D. With this operation, by switching a plurality of enhancement degrees having different degrees of enhancement, it is possible to perform observation and diagnosis with an enhancement effect appropriate for the user.

### Seventh Embodiment

Next, a seventh embodiment will be described. The endoscope system according to the seventh embodiment has a difference from the endoscope system 1D according to the above-described sixth embodiment in configuration and processing to be executed. Accordingly, the following will describe a functional configuration of the endoscope system according to the seventh embodiment and then describe processing to be executed by the endoscope system according to the seventh embodiment. A same reference sign will be given to the configuration identical to the configuration of the endoscope system 1D according to the above-described sixth embodiment, and detailed description thereof will be omitted.

### Functional Configuration of Endoscope System

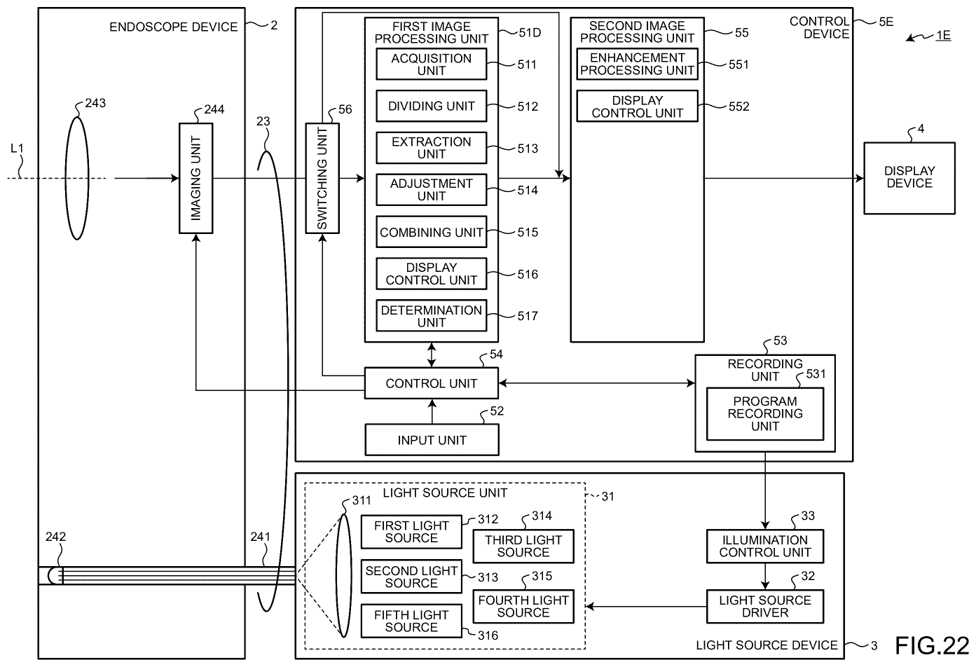

FIG. 22 is a block diagram illustrating a functional configuration of an endoscope system according to the seventh embodiment. An endoscope system 1E illustrated in FIG. 22 includes a control device 5E instead of the control device 5D of the endoscope system 1D according to the above-described sixth embodiment. The control device 5E further includes a switching unit 56 in addition to the functional configuration of the control device 5D according to the sixth embodiment described above.

Under the control of the control unit 54, the switching unit 56 outputs an image signal input from the endoscope device 2 to one of the first image processing unit 51D and the second image processing unit 55. Specifically, under the control of the control unit 54, the switching unit 56 performs operations of inputting image signals such that, in a case where the user operates the input unit 52 and selects processing mode 1, the image signal input from the endoscope device 2 is to be input to the first image processing unit 51D, and in a case where the user operates the input unit 52 and selects processing mode 2, the image signal input from the endoscope device 2 is to be input to the second image processing unit 55.

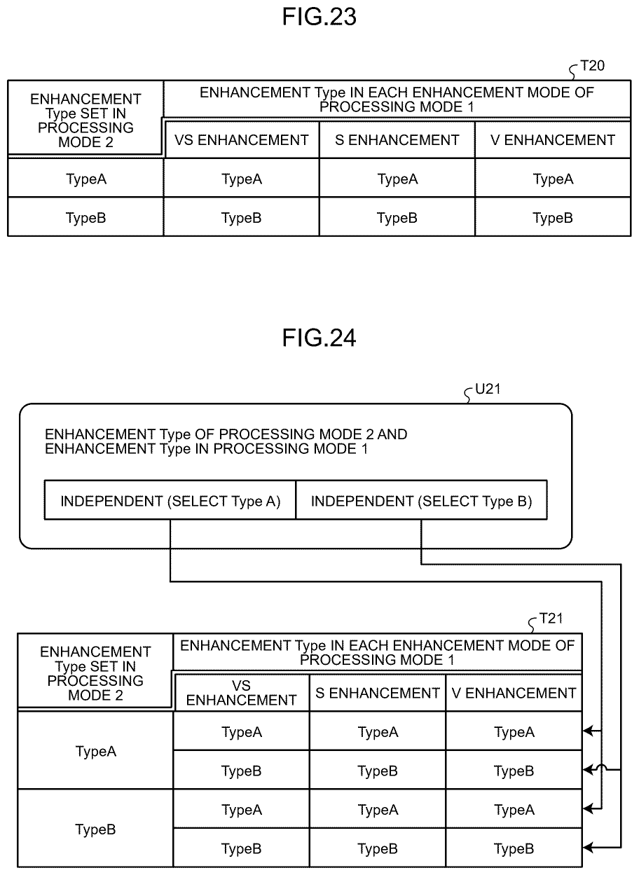

The endoscope system 1E having this configuration operates, under the control of the control unit 54, such that, when the user operates the input unit 52 to select processing mode 1 or processing mode 2, each enhancement mode (V enhancement mode, S enhancement mode, and VS enhancement mode) is to be selected in the selection of processing mode 1, and each enhancement type (Type A, Type B, or Type C) in the second image processing unit 55 is to be selected in the selection of processing mode 2. Processing parameters of the first image processing unit 51D and the second image processing unit 55 in the switching of each processing mode (processing mode 1 and processing mode 2) by the endoscope system 1E will be described. FIG. 23 is a diagram of a parameter table illustrating a relationship between an enhancement type set in processing mode 2 and enhancement mode/enhancement type set in processing mode 1. Note that each enhancement mode indicates any of the V enhancement mode, the S enhancement mode, and the VS enhancement mode.

As illustrated in parameter table T20 of FIG. 23, when the user operates the input unit 52 to select processing mode 2, the enhancement type is set to Type A or Type B, and then processing mode 1 is selected, and thereafter one of the enhancement modes is selected, the control unit 54 reads each processing parameter (refer to a parameter table T20 of FIG. 23) of the first image processing unit 51D and the second image processing unit 55 from the recorded data recorded by the recording unit 53 and changes the processing parameters. Here, Type A and Type B are sharpness enhancement processing with different characteristics. In this manner, the control unit 54 sets the enhancement type of the second image processing unit 55 in processing mode 1 in coordination with the setting of the enhancement type of the second image processing unit 55 in processing mode 2. With this configuration, by switching a plurality of enhancement modes having different degrees in enhancement processing and suppression processing in coordination with enhancement types, it is possible to perform observation and diagnosis with an enhancement effect appropriate for the user.

Note that the enhancement type in processing mode 1 may be set independently of the enhancement type in processing mode 2. FIG. 24 is a diagram illustrating an outline when switching the enhancement type in processing mode 2 and the enhancement type in processing mode 1 independently of each other.

As illustrated in FIG. 24, when the user operates the input unit 52 to select either Independent (Select Type A) or Independent (Select Type B) on setting menu screen U21, and one of the enhancement modes in processing mode 1 is selected, the control unit 54 selects either Type A or Type B for all the enhancement modes and sets the processing parameters of either Type A or Type B (refer to Table T21 in FIG. 24) on the enhancement processing unit 551 of the second image processing unit 55.

In addition, the enhancement type combined with each enhancement mode of processing mode 1 can be selected in coordination with or independently of the enhancement type selected in processing mode 2.

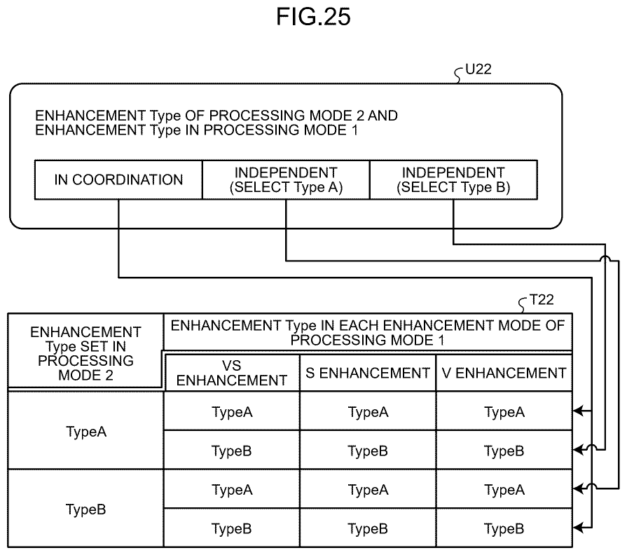

FIG. 25 is a diagram illustrating an outline when switching the enhancement type in processing mode 2 and the enhancement type in processing mode 1 in coordination with or independently of each other.

As illustrated in FIG. 25, when the user operates the input unit 52 to select any one of In coordination, Independent (Select Type A), and Independent (Select Type B) on setting menu screen U22 and then any one of processing mode 1 and enhancement mode is selected, the control unit 54 selects one of enhancement type A and Type B, which has been set for all enhancement modes in selection in processing mode 2 according to the selection, and sets the processing parameters of either Type A or Type B on the enhancement processing unit 551 of the second image processing unit 55.

Furthermore, the enhancement type can be set in combination for each enhancement mode of processing mode 1 independently of the enhancement type set by the user in processing mode 2.

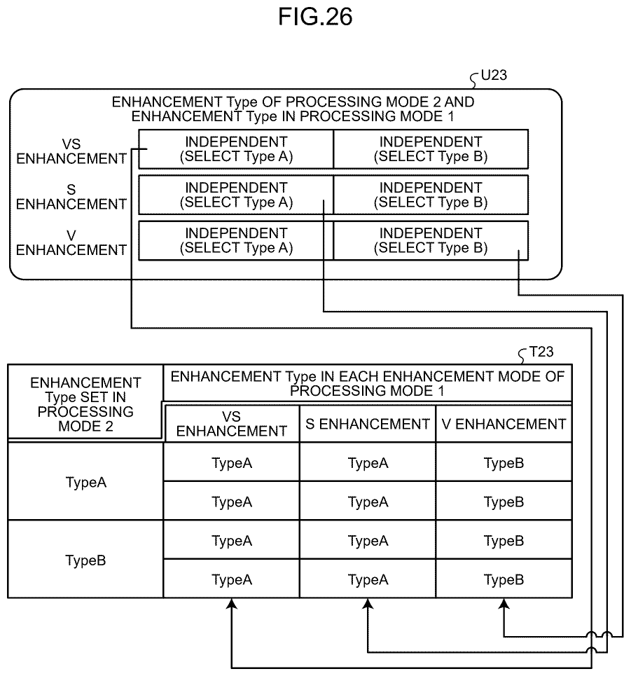

FIG. 26 is a diagram illustrating an outline when switching the enhancement type in processing mode 2 and the enhancement type in processing mode 1 by selecting independently for each enhancement mode.

As illustrated in FIG. 26, when the user operates the input unit 52 and selects either Independent (Select Type A) or Independent (Select Type B) as the enhancement type for each enhancement mode of processing mode 1 on setting menu screen U23, the control unit 54 selects either Type A or Type B for each enhancement mode according to the selection, and sets the processing parameters of either Type A or Type B on the enhancement processing unit 551 of the second image processing unit 55.

In addition, the use can select either the enhancement type to be combined with each enhancement mode of processing mode 1 is to be operated in coordination with or independently of the enhancement type selected in processing mode 2.

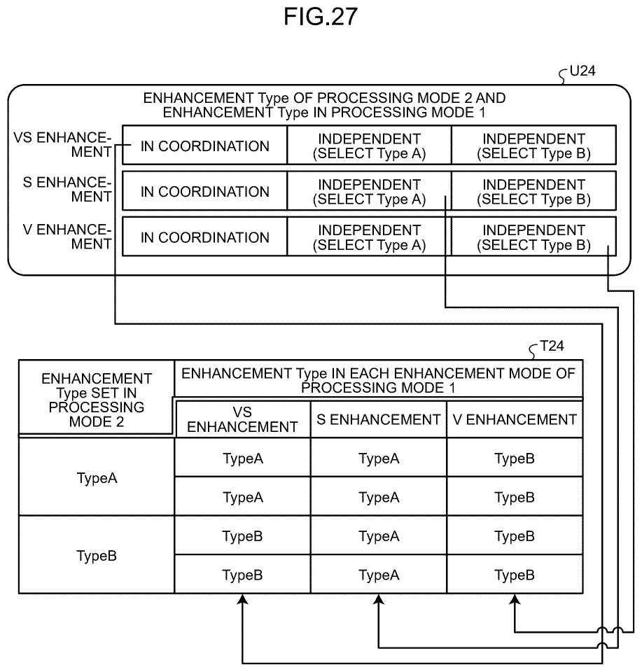

FIG. 27 is a diagram illustrating an outline when the enhancement type in processing mode 2 and the enhancement type in processing mode 1 is switched by selecting the types independently or continuously for each enhancement mode.

As illustrated in FIG. 27, when the user operates the input unit 52 to select one of In coordination, Independent (Select Type A), and Independent (Select Type B) as the enhancement type for each enhancement mode of processing mode 1 on setting menu screen U24, the control unit 54 performs selection in coordination with the enhancement type which has been set for each enhancement modes in selection of processing mode 2 according to the selected item, or selects either Type A or Type B independently, and sets the processing parameters of either Type A or Type B on the enhancement processing unit 551 of the second image processing unit 55.

Although Type A and Type B have been described above as an example, it is also allowable to use a parameter table in which Color enhancement processing Type C is added to options. Furthermore, it is also allowable to use a parameter table in which enhancement type combining sharpness enhancement and color enhancement is added to options.

According to the seventh embodiment described above, observation and diagnosis can be performed with an enhancement effect appropriate for the user.

### Other Embodiments

Furthermore, a plurality of constituents disclosed in the endoscope system according to the above-described first to fifth embodiments of the present disclosure may be appropriately combined to form various embodiments. For example, some constituents may be deleted from all the constituents described in the endoscope system according to the embodiment of the present disclosure described above. Furthermore, the constituents described in the endoscope system according to the embodiment of the present disclosure described above may be appropriately combined.

While the endoscope system according to the first to fifth embodiments of the present disclosure use a wired connection, the connection may be performed wirelessly via a network.

Furthermore, in the first to fifth embodiments of the present disclosure, the functions of the image processing units 51, 51A, 51B, and 51C included in the endoscope system, for example, the functional modules of the acquisition unit 511, the dividing unit 512, the extraction unit 513, the adjustment units 514, 514A, 514B, and 514C, the combining unit 515, the display control unit 516, and the determination unit 517 may be provided in a server connectable via a network, an image processing apparatus capable of bidirectionally communicating with the endoscope system, or the like. Naturally, each functional module may be individually provided in a server, an image processing apparatus, or the like.

The first to fifth embodiments of the present disclosure may have an observation mode corresponding to each of the above-described first to fifth embodiments. In this case, according to the first to fifth embodiments of the present disclosure, the mode may be switched to the observation mode corresponding to each of the above-described first to fifth embodiments in accordance with the operation signal from the input unit 52 or the operation signals from the plurality of switches 223. With this configuration, the user can observe the mucosa and the blood vessel of the biological tissue while selectively enhancing and suppressing each of the desired microstructures and microvessels.

Furthermore, in the endoscope system according to the first to fifth embodiments of the present disclosure, the above-described “unit” can be replaced with “means”, “circuit”, or the like. For example, the control unit is interchangeable with a control means and a control circuit.

In the flowcharts in this description, context of the processes among the steps is described by using expressions such as “first”, “thereafter”, and “subsequently”, but the sequences of the processes needed for implementing the embodiments are not intended to be uniquely defined by these expressions. In other words, the order of processing in the flowcharts described herein can be changed within a range implementable without contradiction.

While some embodiments of the present application have been described in detail with reference to the drawings, these embodiments are provided for an exemplifying purpose. The present invention can be implemented in other modes to which various modifications and enhancements are applied based on the knowledge of those who are skilled in the art including the modes described in the summary of the present disclosure.

According to the present disclosure, it is possible to selectively enhance and suppress a microstructure and a microvessel in an image regardless of an observation distance.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing apparatus comprising a processor configured to:
irradiate a biological tissue including a microstructure and a microvessel with illumination light including blue-violet narrow band light, and acquire an image signal generated by capturing return light from the biological tissue;
extract local contrast information in the image signal; and
perform one or more of enhancement processing or suppression processing on the image signal based on the local contrast information so as to generate a display image, the enhancement processing being processing of enhancing at least one of microstructure information related to the microstructure in the biological tissue or microvessel information related to the microvessel in the biological tissue, the suppression processing being processing of suppressing at least one of the microstructure information related to the microstructure in the biological tissue or the microvessel information related to the microvessel in the biological tissue.

2. The image processing apparatus according to claim 1,
wherein the processor is further configured to:
extract, for each of pixels constituting an input image corresponding to the image signal, a local contrast value as the local contrast information;
determine, for each of the pixels, whether the local contrast value is equal to or larger than at least one reference value preset; and
perform one of the enhancement processing or the suppression processing on a signal value of a pixel having the local contrast value equal to or larger than the reference value, and perform the other of the enhancement processing and the suppression processing on a signal value of a pixel having the local contrast value not equal to or larger than the reference value, so as to generate the display image.

3. The image processing apparatus according to claim 2,
wherein the microstructure information corresponds to the pixel of the signal value having the local contrast value equal to or larger than the reference value.

4. The image processing apparatus according to claim 2,
wherein the microvessel information corresponds to the pixel of the signal value having the local contrast value not equal to or larger than the reference value.

5. The image processing apparatus according to claim 3,
wherein the enhancement processing is processing of setting the local contrast value away from the reference value, and
the processor performs the enhancement processing on the microstructure information.

6. The image processing apparatus according to claim 4,
wherein the suppression processing is processing of bringing the local contrast value closer to the reference value, and
the processor performs the suppression processing on the microvessel information.

7. The image processing apparatus according to claim 3,
wherein the suppression processing is processing of bringing the local contrast value closer to the reference value, and
the processor performs the suppression processing on the microstructure information.

8. The image processing apparatus according to claim 7,
wherein the enhancement processing is processing of setting the local contrast value away from the reference value, and
the processor performs the enhancement processing on the microvessel information.

9. The image processing apparatus according to claim 1,
wherein the processor is further configured to extract the local contrast information based on a relative signal strength ratio between a signal value of a target pixel in an input image corresponding to the image signal and a signal value of a surrounding pixel of the target pixel.

10. The image processing apparatus according to claim 1,
wherein the processor is further configured to:
divide the image signal into an illumination light component and a reflectance component; and
increase or decrease a signal amplitude value of the reflectance component.

11. The image processing apparatus according to claim 1,
wherein the processor is further configured to:
divide the image signal into an illumination light component and a reflectance component; and
increase or decrease a signal amplitude value of the illumination light component.

12. The image processing apparatus according to claim 1,
wherein the processor is further configured to:
divide the image signal into an illumination light component and a reflectance component;
perform at least one of the enhancement processing or the suppression processing on a signal amplitude value of the reflectance component; and
combine the reflectance component that has undergone the at least one of the enhancement processing or the suppression processing, and the illumination light component, to generate the display image.

13. The image processing apparatus according to claim 1,
wherein the processor is further configured to:
divide the image signal into an illumination light component and a reflectance component;
perform at least one of the enhancement processing or the suppression processing on the reflectance component;
perform gain adjustment processing of performing gain adjustment on the illumination light component; and
combine the illumination light component that has undergone the gain adjustment processing and the reflectance component that has undergone the at least one of the enhancement processing or the suppression processing, so as to generate the display image.

14. The image processing apparatus according to claim 1,
wherein the processor is further configured to:
generate two illumination light components having mutually different frequency bands based on the image signal;
generate two reflectance components based on each of the two illumination light components and the image signal;
combine the two reflectance components by a predetermined coefficient;
perform at least one of the enhancement processing or the suppression processing on a combining result obtained by combining the two reflectance components by the predetermined coefficient; and
combine a result of performing the at least one of the enhancement processing or the suppression processing with one of the two illumination light components, so as to generate the display image.

15. The image processing apparatus according to claim 1,
wherein the processor is further configured to, based on a signal value of each target pixel included in the image signal and a signal value of each of a plurality of surrounding pixels in the target pixel, extract a contrast value that is a relative signal strength ratio, as the local contrast information for each pixel.

16. The image processing apparatus according to claim 1,
wherein, based on the local contrast information, the processor is configured to determine, for each pixel, whether a local contrast value is equal to or larger than a reference value, and extract the microstructure information and the microvessel information.

17. The image processing apparatus according to claim 1,
wherein, based on the local contrast information, the processor is configured to extract a pixel having a local contrast value larger than a reference value as the microstructure information, and extract a pixel having the local contrast value smaller than a reference value as the microvessel information.

18. The image processing apparatus according to claim 1,
wherein the processor is configured to determine, for each pixel, whether a local contrast value is equal to or larger than a first reference value, and extract the microstructure information.

19. The image processing apparatus according to claim 1,
wherein the processor is configured to extract a pixel having a local contrast value larger than at least a first reference value as the microstructure information.

20. The image processing apparatus according to claim 1,
wherein the processor is configured to determine, for each pixel, whether a local contrast value is equal to or smaller than a second reference value, and extract the microvessel information.

21. The image processing apparatus according to claim 1,
wherein the processor is configured to extract a pixel having a local contrast value smaller than at least a second reference value as the microvessel information.

22. The image processing apparatus according to claim 1,
wherein the processor is capable of selecting an enhancement mode.

23. The image processing apparatus according to claim 22,
wherein the enhancement mode includes: a first enhancement mode of enhancing the microvessel; a second enhancement mode of enhancing the microstructure; and a third enhancement mode of enhancing the microvessel and the microstructure.

24. The image processing apparatus according to claim 23,
wherein, when the first enhancement mode is set as the enhancement mode, the processor is configured to extract at least the microvessel information based on the local contrast information and apply the enhancement processing to the microvessel information.

25. The image processing apparatus according to claim 23,
wherein, when the second enhancement mode is set as the enhancement mode, the processor is configured to extract at least the microstructure information based on the local contrast information and apply the enhancement processing to the microstructure information.

26. The image processing apparatus according to claim 23,
wherein, when the third enhancement mode is set as the enhancement mode, the processor is configured to extract the microvessel information and the microstructure information based on the local contrast information and apply the enhancement processing to the microvessel information and the microstructure information.

27. The image processing apparatus according to claim 1,
wherein a correspondence relationship between an input value before an application of the enhancement processing regarding a local contrast value and an output value after the application of the enhancement processing regarding the local contrast value is stored in a memory.

28. The image processing apparatus according to claim 1,
wherein a correspondence relationship between an input value before an application of the suppression processing regarding a local contrast value and an output value after the application of the suppression processing regarding the local contrast value is stored in a memory.

29. The image processing apparatus according to claim 1,
wherein a correspondence relationship between an input value before an application of the enhancement processing regarding a local contrast value related to the microvessel information and an output value after the application of the enhancement processing regarding the local contrast value related to the microvessel information is stored in a memory.

30. The image processing apparatus according to claim 1,
wherein a correspondence relationship between an input value before an application of the enhancement processing regarding a local contrast value related to the microstructure information and an output value after the application of the enhancement processing regarding the local contrast value related to the microstructure information is stored in a memory.

31. The image processing apparatus according to claim 1,
wherein the processor is capable of selecting an enhancement level.

32. The image processing apparatus according to claim 31,
wherein the enhancement level includes a first enhancement level and a second enhancement level.

33. The image processing apparatus according to claim 32,
wherein the first enhancement level applies enhancement processing higher in intensity than the second enhancement level.

34. The image processing apparatus according to claim 33,
wherein the enhancement processing is processing of setting a local contrast value away from a reference value, and the first enhancement level sets the local contrast value away from the reference value with a value greater than in the second enhancement level.

35. A medical system comprising a light source device, an imaging device, and a medical device,
wherein the light source device includes a light source configured to irradiate a biological tissue including a microstructure and a microvessel with illumination light including blue-violet narrow band light,
the imaging device includes an image sensor configured to generate an image signal by capturing return light from the biological tissue,
the medical device includes a processor configured to:
acquire the image signal;
extract local contrast information in the image signal;
perform one or more of enhancement processing or suppression processing on the image signal based on the local contrast information so as to generate a display image, the enhancement processing being processing of enhancing at least one of microstructure information related to a microstructure in the biological tissue or microvessel information related to a microvessel in the biological tissue, the suppression processing being processing of suppressing at least one of the microstructure information related to the microstructure in the biological tissue or the microvessel information related to the microvessel in the biological tissue.

36. An operation method of an image processing apparatus, the image processing apparatus including a processor, the method to be performed by the processor,
the method comprising:
controlling a light source to emit at least blue-violet light and acquiring an image signal generated at emission of the blue-violet light;
extracting local contrast information in the image signal; and
performing one or more of enhancement processing or suppression processing on the image signal based on the local contrast information so as to generate a display image, the enhancement processing being processing of enhancing at least one of microstructure information related to a microstructure in a biological tissue or microvessel information related to a microvessel in the biological tissue, the suppression processing being processing of suppressing at least one of microstructure information related to the microstructure in the biological tissue or microvessel information related to the microvessel in the biological tissue.

37. A non-transitory computer-readable recording medium with an executable program stored thereon, the program causing a processor to execute:
irradiating a biological tissue including a microstructure and a microvessel with illumination light including blue-violet narrow band light, and acquiring an image signal generated by capturing return light from the biological tissue;
extracting local contrast information in the image signal; and
performing one or more of enhancement processing or suppression processing on the image signal based on the local contrast information so as to generate a display image, the enhancement processing being processing of enhancing at least one of microstructure information related to the microstructure in the biological tissue or microvessel information related to the microvessel in the biological tissue, the suppression processing being processing of suppressing at least one of the microstructure information related to the microstructure in the biological tissue or the microvessel information related to the microvessel in the biological tissue.
